(19) United States
(12) Patent Application Publication
(10) Pub. No.: US 2022/0204984 A1
(43) Pub. Date: Jun. 30, 2022

(54) METHODS AND COMPOSITIONS FOR MODIFYING PHENOTYPES OF PLANTS EXPRESSING FATTY ACID TRANSGENES AND REDUCED EXPRESSION OF BADC GENES

(71) Applicants: Brookhaven Science Associates, LLC, Upton, NY (US); The Research Foundation For The State University of New York, Albany, NY (US)

(72) Inventors: John Shanklin, Shoreham, NY (US); Xioa-Hong Yu, Mount Sinai, NY (US); Hui Liu, Middle Island, NY (US); Jantana Keereetaweep, Wading River, NY (US); Yuanheng Cai, Shirley, NY (US)

(21) Appl. No.: 17/534,993

(22) Filed: Nov. 24, 2021

Related U.S. Application Data
(60) Provisional application No. 63/131,755, filed on Dec. 29, 2020.
(60) Provisional application No. 63/118,209, filed on Nov. 25, 2020.

(51) Int. Cl. C12N 15/82

(52) U.S. Cl. CPC C12N 15/8247

(57) ABSTRACT

Compositions that are plants, seeds or crops that have a combination of defective BADC genes and genes for making hydroxy fatty acids produced normal levels of oil containing specialty fatty acids, and exhibited an increases in total oil accumulation, increase in absolute hydroxy (specialized) fatty acid accumulation per seed and/or per plant and/or per unit land area. Defective BADC genes and genes for synthesizing hydroxy fatty acids are combined to produce specialized fatty acids without substantially slowing production of endogenous fatty acids. Methods are also described for increasing production of unusual fatty acids and increasing total fatty acid levels in plants by a mechanism involving combining defective BADC genes and genes for making hydroxy fatty acids to produce steady or increased levels of oil containing the increased specialty products as described herein.

### CROSS-REFERENCE TO RELATED APPLICATIONS

This application is a Non-Provisional application which claims benefit of U.S. Patent Application Ser. No. 63/131,755, filed on Dec. 29, 2020, now expired, which is herein incorporated by reference in its entirety.

### GOVERNMENT SUPPORT

This invention was made with Government support under contract numbers DE-SC0012704 and DE-SC0018420 awarded by the U.S. Department of Energy and IOS-13-39385 awarded by the National Science Foundation. The Government has certain rights in the invention.

### SEQUENCE

A Sequence Listing has been submitted in an ASCII text file named “19963.txt” created on Mar. 23, 2022, consisting of 101,032 bytes, the entire content of which is herein incorporated by reference.

### FIELD OF THE INVENTION

BADC (biotin attachment domain-containing) mutants specifically badc1, badc3 for beneficial effects on unusual fatty acid (also known as specialized fatty acid accumulation).

### SUMMARY OF THE INVENTION

When enzymes that convert common fatty acids to unusual fatty acids (hydroxy, epoxy, conjugated or cyclopropane fatty acids) are expressed in plants like Arabidopsis or Camelina, the unusual fatty acid accumulates but the total yield of fatty acids decreases. Mutations in the badc1,badc3 genes i.e., negative regulators of acetyl CoA carboxylase (ACCase) may mitigate this effect and restore or maintain total fatty acid levels thereby facilitating the accumulation of unusual fatty acids.

The present discovery may be a way to reverse a roadblock in plants to specialty oil production thereby providing a pathway to grow crops that produce industrially important high-value fatty acids. Hundreds of naturally occurring specialty fatty acids (building blocks of oils) may have potential for use as raw materials for making for example, lubricants, plastics, or pharmaceuticals, if they can be produced at large scale by crop plants. Prior attempts to put genes for making these specialty building blocks into crops have resulted in the adverse effect, namely, transgenic seeds making the specialty fatty acids experienced a reduction in their oil accumulation.

The mechanism behind the oil-production slowdown is described herein. Model plants were crossbred and detailed biochemical-genetic analyses were conducted that demonstrate a strategy for reversing the roadblock and increasing production. This may provide potential for making at least one or more industrially important specialty fatty acid in plants, crops or seeds.

While the genes responsible for making specialty fatty acids were discovered several decades ago, this present mechanism may allow them to be put into compositions such as plants, crops or seeds to make renewable sources of desired fatty acids without slowing fatty acid and oil synthesis. The study focused on challenges associated with specialized fatty acid production in plants, and on deciphering the biochemical feedback loop that plants use to regulate ordinary or regular fatty acid (FA) and oil production. This study led to the discovery of a mechanism by which plants down-regulate oil synthesis when levels of a plant's ordinary or regular (endogenous) fatty acids (FAs) get too high. In other words, the system operates or functions like a thermostat. When endogenous FA (heat) gets above a certain set point, the system (furnace) turns off.

With plant oils, the machinery that controls production is an enzyme called ACCase (acetyl coA carboxylase). It has four parts, or subunits: biotin carboxylase (BC), biotin carboxyl carrier protein (BCCP), and two carboxyltransferases, α-carboxyltransferase and β-carboxyltransferase. As long as endogenous fatty acids are below a certain level, the four subunits act coordinately to convert acetyl-CoA to malonyl-CoA. But feeding plants additional endogenous fatty acids triggers a substitution in the machinery in which the BCCP subunit is gets replaced by biotin attachment domain-containing protein (BADC), a homolog of BADC that lacks a critical biotin attachment amino acid and is therefore inactive. ACCase in which BCCP has been replaced by BADC slows the production of malonyl-CoA and therefor the synthesis of fatty acids. In contrast, the shutdown mechanism triggered by the accumulation of specialty fatty acids (ones being produced by genes expressed in the plant) kicks in when even small amounts of the “foreign” fatty acids are present, and endogenous fatty acids aren't in excess. Because of this, they appeared to be two separate processes. But it was speculated whether the specialty fatty acids were triggering the same off switch triggered by high levels of ordinary fatty acids.

In a strain of Arabidopsis (a model plant) with two of its BADC genes deleted, the downregulation of ACCase is disabled and the plants make high levels of endogenous fatty acids. Further study looked at what would happen if the BADC genes were disabled in plants engineered to produce specialty fatty acids. A research strategy was designed to crossbreed the defective off-switch plants with an Arabidopsis strain engineered to produce hydroxy fatty acids-one of the specialty types scientists would like to produce for industrial applications. This latter strain could make the hydroxy fatty acids, but its rate of oil synthesis was only half that of normal (unmodified) plants and it accumulated significantly less oil in its seeds.

When crossing four separate genetic factors (two mutant BADC genes, a mutant in fatty acid elongase 1 (FAE1) and an overexpression of the Ricinus communis 12-hydroxylase gene (FAH), it takes several plant generations to produce and identify plants homozygous for the four desired genes (see FIGS. 16A and 16B FAH sequences; FIGS. 16O and 16P FAE1 sequences; FIGS. 16U and 16V mutant FAE1 sequences). Polymerase chain reaction (PCR) tests were run for analyses of greater many hundreds of plants to find those homozygous for all four alleles. Those plants were biochemically characterized, to compare their rates of ACCase activity with those of the two parental Arabidopsis lines used to make the new genetic combinations. Plants that had the combination of defective BADC genes and genes required for making hydroxy fatty acids produced normal (unaltered) levels of oil containing the specialty products. Compared with plants that had normal (wild-type) BADC genes, the new plants exhibited increases in the total amount of fatty acid per seed, the total seed oil content per plant, and the seed yield per plant. The BADC-defective plants were unresponsive to the presence of hydroxy fatty acids and the usual response of turning off the ACCase was gone. The results prove that BADC is the mechanism for reducing ACCase activity in both scenarios—the accumulation of excess endogenous fatty acids and the presence of hydroxy fatty acid.

The BADC mechanism may be specific to the accumulation of hydroxy fatty acids, or may be common to other ‘foreign’ fatty acids that also reduce ACCase activity. BADC may be a general mechanism, and mutations that reduce their activity may allow for the accumulation of additional specialty fatty acids in oil-rich seeds of crop plants with minimal reduction of oil yield. This fundamental mechanistic understanding of biochemical regulation may be useful towards a viable, sustainable bioeconomy. This approach may also be used to make valuable renewable industrial starting materials at low cost in plants from carbon dioxide and sunlight, rather than relying on petrochemicals.

Thus, in one embodiment, the invention provides a composition comprising one or more mutated BADC genes for accumulating unusual fatty acids and maintaining or increasing total fatty acid levels, oil content in the composition as described herein.

The invention also provides a composition produced by any one or more of the methods of the invention, wherein the composition is a seed, plant or crop.

The invention also provides a plant composition comprising a combination of defective BADC genes and genes for synthesizing hydroxy fatty acids for producing specialized fatty acids without slowing production of endogenous fatty acids as described herein.

The invention also provides a method of increasing production of unusual fatty acids and increasing total fatty acid levels in plants, crops or seeds by a mechanism involving combining defective BADC genes and genes for making hydroxy fatty acids to produce steady or increased levels of oil containing the specialty products as described herein.

In one embodiment, the invention provides a method of modifying a plant or part thereof, comprising producing a transgenic plant or part thereof that comprises a transgenic plant cell, said transgenic plant cell comprising a) reduced (e.g., lacks) expression of one or both of endogenous BADC1 and BADC3 genes, and b) expression of one or more transgenes that alters metabolism of a target fatty acid. In one embodiment, said transgenic plant exhibits one or more phenotypes of a) an increased amount of total seed fatty acid per plant, b) improved establishment of one or both of roots and plant aerial parts, and c) rescued or increased seed yield per plant. In one embodiment, said transgenic plant produces seeds, said seeds exhibiting one or more of rescued or increased seed germination rate, rescued or increased amount of total seed fatty acid per seed, rescued or increased amount of said target fatty acid per seed, and rescued or increased proportion of said target fatty acid relative to said total seed fatty acid per seed. In one embodiment, said transgenic plant cell comprises wild type BADC1 gene and reduced (or lacks) expression of said endogenous BADC3 gene. In one embodiment, said transgenic plant cell comprises wild type BADC1 gene and lacks expression of said endogenous BADC3 gene. In one embodiment, said producing comprises deleting at least a portion of said wild type BADC3 gene. In one embodiment, said deleting comprises using clusters of regularly interspaced short palindromic repeats (CRISPR) gene editing. In one embodiment, said transgenic plant cell lacks an alteration in one or both the enzyme activity and protein expression level of wild type acetyl CoA carboxylase (ACCase). In one embodiment, said target fatty acid comprises a foreign fatty acid that is not naturally produced in a wild type of said cell. In one embodiment, said target fatty acid comprises one or more of hydroxyl fatty acids, medium-chain fatty acids, very-long-chain fatty acids (VLCFAs), monounsaturated fatty acids (MUFAs), γ-linolenic acid, stearidonic acids, α-eleostearic acid, conjugated fatty acids, expoxy fatty acids, cyclic fatty acids and acetylenic fatty acids. In one embodiment, said target fatty acid comprises a hydroxyl fatty acid exemplified by ricinoleic acid. In one embodiment, said plant cell is selected from a Camelina sativa plant cell, a Brassica napus plant cell and Glycine max plant cell. In one embodiment, said transgenic plant cell comprises a genomic mutation such as fad2, fad3 and fae1 or any combination thereof. In one embodiment, said genomic mutation is selected from fad2/fae1 and fad3/fae1. In one embodiment, said transgenic plant cell is a cell from Arabidopsis thaliana and comprises genomic mutation fad2/fae1. In one embodiment, said transgenic plant cell is a cell from a plant selected from Camelina sativa, Brassica napus and Glycine max, and comprises a genomic mutation fad3/fae1. In one embodiment, said transgenic plant cell is a cell from a plant selected from Camelina sativa, Brassica napus and Glycine max, and comprises a genomic mutation fad3/fae1, wherein said transgene that alters metabolism of said target fatty acid encodes one or more of acetylanase, conjugase and epoxygenase. In one embodiment, said transgene that alters metabolism of said target fatty acid comprises a transgene encoding fatty acid hydroxylase exemplified by Ricinus fatty acid hydroxylase (RcFAH) mutant fatty acid elongation 1 (FAE1) (see exemplary FIGS. 16A and 16B FAH sequences; FIGS. 16O and 16P FAE1 sequences; FIGS. 16U and 16V mutant FAE1 sequences), E. coli Cyclopropane fatty acid synthase (see exemplary FIGS. 17 A and 17 B), epoxygenase exemplified by Crepis palaestina delta 12 fatty acid epoxygenase (see exemplary FIGS. 17 C and 17 D), acetylenase exemplified by Crepis alpina delta-12 fatty acid acetylenase (see exemplary FIGS. 17 E and 17 F), conjugase exemplified by Momordica charantia Conjugase (FadX) (see exemplary FIG. 17 G). In one embodiment, said transgene comprises a transgene encoding Ricinus fatty acid hydroxylase (FAH). In one embodiment, said transgene comprises a transgene encoding E. coli cyclopropane fatty acid synthase. In one embodiment, said one or more transgene that alters metabolism of said target fatty acid is under control of a seed-specific promoter. In one embodiment, said transgenic plant or part thereof that comprises reduced (or lacks) expression of one or both of said endogenous BADC1 and BADC3 genes contains a mutation in said one or both of said endogenous BADC1 and BADC3 genes. In one embodiment, said transgenic plant or part thereof is homozygous for said mutation in said one or both of said endogenous BADC1 and BADC3 genes. In one embodiment, said transgenic plant or part thereof is homozygous null for said one or both of said endogenous BADC1 and BADC3 genes. In one embodiment, said transgenic plant or part thereof is homozygous for said one or more transgene that alters metabolism of said target fatty acid. In one embodiment, said transgenic plant or part thereof is heterozygous for said mutation in said one or both of said endogenous BADC1 and BADC3 genes. In one embodiment, said transgenic plant or part thereof is heterozygous for said one or more transgene that alters metabolism of said target fatty acid. In one embodiment, said transgenic plant cell or part thereof is stably transformed with said transgene that alters metabolism of said target fatty acid. In one embodiment, said producing comprises transforming a plant cell with one or more recombinant nucleotide sequences that partially or totally silence of one or both of said endogenous BADC1 and BADC3 genes. In one embodiment, said producing comprises transforming said plant cell with one or more recombinant nucleotide sequences that alter metabolism of said target fatty acid. In one embodiment, said producing comprises transforming said plant cell with one or more recombinant nucleotide sequences that partially or totally silence of one or both of said endogenous BADC1 and BADC3 genes. In one embodiment, said producing comprises transforming said plant cell with one or more recombinant nucleotide sequences that (a) partially or totally silence of one or both of said endogenous BADC1 and BADC3 genes, and (b) alter metabolism of said target fatty acid. In one embodiment, said producing comprises crossing a first transgenic plant comprising said reduced or lacking expression of one or both of said endogenous BADC1 and BADC3 genes to a second transgenic plant comprising said one or more transgene that alters metabolism of said target fatty acid.

In one embodiment, the present invention provides a method of modifying a plant or part thereof, comprising producing a transgenic plant or part thereof that comprises a transgenic plant cell, said transgenic plant cell comprising a) reduced expression of one or both of endogenous BADC1 and BADC3 genes, and b) expression of one or more transgenes that alters metabolism of a target fatty acid. In one embodiment, said transgenic plant exhibits one or more phenotypes of a) increased amount of total seed fatty acid per plant, b) improved establishment of one or both of roots and plant aerial parts, and c) rescued or increased seed yield per plant. In one embodiment, said transgenic plant produces seeds, said seeds exhibiting one or more of rescued or increased seed germination rate, rescued or increased amount of total seed fatty acid per seed, rescued or increased amount of said target fatty acid per seed, and rescued or increased proportion of said target fatty acid relative to said total seed fatty acid per seed. In one embodiment, said transgenic plant cell comprises wild type BADC1 gene and reduced expression of said endogenous BADC3 gene. In one embodiment, said transgenic plant cell comprises wild type BADC1 gene and lacks expression of said endogenous BADC3 gene. In one embodiment, said producing comprises deleting at least a portion of said wild type BADC3 gene. In one embodiment, said deleting comprises using clusters of regularly interspaced short palindromic repeats (CRISPR) gene editing. In one embodiment, said transgenic plant cell lacks an alteration in one or both the enzyme activity and protein expression level of wild type acetyl CoA carboxylase (ACCase). In one embodiment, said target fatty acid comprises a foreign fatty acid that is not naturally produced in a wild type of said cell. In one embodiment, said target fatty acid comprises one or more of hydroxyl fatty acids, medium-chain fatty acids, very-long-chain fatty acids (VLCFAs), monounsaturated fatty acids (MUFAs), 7-linolenic acid, stearidonic acids, α-eleostearic acid, conjugated fatty acids, expoxy fatty acids, cyclic fatty acids and acetylenic fatty acids. In one embodiment, said target fatty acid comprises a hydroxyl fatty acid. In one embodiment, said hydroxyl fatty acid comprises ricinoleic acid. In one embodiment, said plant cell is selected from a Camelina sativa plant cell, a Brassica napus plant cell and Glycine max plant cell. In one embodiment, said transgenic plant cell comprises a genomic mutation such as fad2, fad3 and fae1 or any combination thereof. In one embodiment, said genomic mutation is selected from fad2/fae1 and fad3/fae1. In one embodiment, said transgenic plant cell is a cell from Arabidopsis thaliana and comprises genomic mutation fad2/fae1. In one embodiment, said transgenic plant cell is a cell from a plant selected from Camelina sativa, Brassica napus and Glycine max, and comprises a genomic mutation fad3/fae1. In one embodiment, said transgenic plant cell is a cell from a plant selected from Camelina sativa, Brassica napus and Glycine max, and comprises a genomic mutation fad3/fae1, wherein said transgene that alters metabolism of said target fatty acid encodes one or more of acetylanase, conjugase and epoxygenase. In one embodiment, said transgene that alters metabolism of said target fatty acid comprises a transgene encoding Ricinus fatty acid hydroxylase (FAH), mutant fatty acid elongation 1 (FAE1), E. coli Cyclopropane fatty acid synthase, Crepis palaestina delta 12 fatty acid epoxygenase, Crepis alpina delta-12 fatty acid acetylenase, Momordica charantia Conjugase (FadX), RcFAH, cyclopropane fatty acid synthase. In one embodiment, said transgene comprises a transgene encoding Ricinus fatty acid hydroxylase (FAH). In one embodiment, said transgene comprises a transgene encoding E. coli cyclopropane fatty acid synthase. In one embodiment, said one or more transgene that alters metabolism of said target fatty acid is under control of a seed-specific promoter. In one embodiment, said transgenic plant or part thereof that comprises reduced expression of one or both of said endogenous BADC1 and BADC3 genes contains a mutation in said one or both of said endogenous BADC1 and BADC3 genes. In one embodiment, said transgenic plant or part thereof is homozygous for said mutation in said one or both of said endogenous BADC1 and BADC3 genes. In one embodiment, said transgenic plant or part thereof is homozygous null for said one or both of said endogenous BADC1 and BADC3 genes. In one embodiment, said transgenic plant or part thereof is homozygous for said one or more transgene that alters metabolism of said target fatty acid. In one embodiment, said transgenic plant or part thereof is heterozygous for said mutation in said one or both of said endogenous BADC1 and BADC3 genes. In one embodiment, said transgenic plant or part thereof is heterozygous for said one or more transgene that alters metabolism of said target fatty acid. In one embodiment, said transgenic plant cell or part thereof is stably transformed with said transgene that alters metabolism of said target fatty acid. In one embodiment, said producing comprises transforming a plant cell with one or more recombinant nucleotide sequences that partially or totally silence of one or both of said endogenous BADC1 and BADC3 genes. In one embodiment, said producing comprises transforming said plant cell with one or more recombinant nucleotide sequences that alter metabolism of said target fatty acid. In one embodiment, said producing comprises transforming said plant cell with one or more recombinant nucleotide sequences that partially or totally silence of one or both of said endogenous BADC1 and BADC3 genes. In one embodiment, said producing comprises transforming said plant cell with one or more recombinant nucleotide sequences that (a) partially or totally silence of one or both of said endogenous BADC1 and BADC3 genes, and (b) alter metabolism of said target fatty acid. In one embodiment, said producing comprises crossing a first transgenic plant comprising said reduced expression of one or both of said endogenous BADC1 and BADC3 genes to a second transgenic plant comprising said one or more transgene that alters metabolism of said target fatty acid.

The invention also provides a transgenic plant or part thereof that comprises a transgenic plant cell that comprises a) reduced or lacks expression of one or both of said endogenous BADC1 and BADC3 genes, and b) one or more transgene that alters metabolism of said target fatty acid. In one embodiment, the transgenic plant exhibits one or more phenotype of a) increased amount of total seed fatty acid per plant, b) improved establishment of one or both of roots and plant aerial parts, and c) rescued or increased seed yield per plant. In one embodiment, the transgenic plant produces seeds, said seeds exhibiting one or more of rescued or increased seed germination rate, rescued or increased amount of total seed fatty acid per seed, rescued or increased amount of said target fatty acid per seed, and rescued or increased proportion of said target fatty acid relative to said total seed fatty acid per seed. In one embodiment, the transgenic plant or part thereof is produced by any one or more of the invention's methods.

In one embodiment, the present invention provides a transgenic plant or part thereof that comprises a transgenic plant cell, wherein said plant cell comprises a) reduced or lacks expression of one or both of said endogenous BADC1 and BADC3 genes, and b) one or more transgene that alters metabolism of a target fatty acid. In one embodiment, said transgenic plant exhibits one or more phenotype of a) increased amount of total seed fatty acid per plant, b) improved establishment of one or both of roots and plant aerial parts, and c) rescued or increased seed yield per plant. In one embodiment, said transgenic plant produces seeds, said seeds exhibiting one or more of rescued or increased seed germination rate, rescued or increased amount of total seed fatty acid per seed, rescued or increased amount of said target fatty acid per seed, and rescued or increased proportion of said target fatty acid relative to said total seed fatty acid per seed.

In one embodiment, the present invention provides a transgenic plant or part thereof that comprises a transgenic plant cell, wherein said plant cell comprises a) reduced or lack of expression of one or both of said endogenous BADC1 and BADC3 genes, and b) one or more transgene that alters metabolism of a target fatty acid.

In one embodiment, the present invention provides a transgenic plant or part thereof that comprises: a) a genomic mutation selected from the group consisting of a mutation of fad2, fad3, and fae1, or any combination of such mutations, b) the reduced expression of one or both endogenous BADC1 and BADC3 genes, and c) one or more transgenes that alter metabolism of a target fatty acid. In one embodiment, the present invention provides a transgenic plant or part thereof that comprises: a) genomic mutation of fad2, fad3, fae1 or any combination thereof, b) reduced expression of one or both endogenous BADC1 and BADC3 genes, and c) one or more transgenes that alter metabolism of a target fatty acid. In one embodiment, said transgenic plant part comprises wild type BADC1 gene and reduced expression of said endogenous BADC3 gene. In one embodiment, said transgenic plant part comprises reduced expression of said endogenous BADC1 gene and BADC3 gene. In one embodiment, said plant is Camelina sativa, Brassica napus or Glycine max. In one embodiment, said genomic mutation is fad2/fae1 or fad3/fae1. In one embodiment, said one or more transgenes encode Ricinus fatty acid hydroxylase (FAH), E. coli cyclopropane fatty acid synthase, Crepis palaestina delta 12 fatty acid epoxygenase, Crepis alpina delta-12 fatty acid acetylenase, or Momordica charantia Conjugase (FadX). In one embodiment, said one or more transgenes are under control of a seed-specific promoter. In one embodiment, said target fatty acid comprises one or more of hydroxyl fatty acids, medium-chain fatty acids, very-long-chain fatty acids (VLCFAs), monounsaturated fatty acids (MUFAs), gamma-linolenic acid, stearidonic acids, alpha-eleostearic acid, conjugated fatty acids, epoxy fatty acids, cyclic fatty acids and acetylenic fatty acids. In one embodiment, said transgenic plant part is from Camelina sativa, Brassica napus or Glycine max, said genomic mutation is fad3/fae1, and said transgene encodes acetylanase, conjugase, epoxygenase or any combinations thereof. In one embodiment, said transgenic plant part is from Camelina sativa, Brassica napus or Glycine max, said genomic mutation is fad2/fae1, and said transgene encodes Ricinus fatty acid hydroxylase. In one embodiment, said transgenic plant part is from Camelina sativa, Brassica napus or Glycine max, said genomic mutation is fae1, and said transgene encodes Ricinus fatty acid hydroxylase. In one embodiment, said reduced expression comprises complete silencing. In one embodiment, said reduced expression comprises complete silencing. In one embodiment, said reduced expression comprises complete silencing.

The invention further provides a progeny plant of the any of the transgenic plants of the invention.

In one embodiment, the present invention provides a method of modifying a plant or part thereof, comprising producing a transgenic plant or part thereof that comprises a transgenic plant cell, said transgenic plant cell comprising a) reduced expression of one or both of endogenous BADC1 and BADC3 genes, and b) expression of one or more transgenes that alters metabolism of a target fatty acid.

In one embodiment, the present invention provides a progeny plant of a transgenic plant or part thereof that comprises a transgenic plant cell, wherein said plant cell comprises a) reduced or lacks expression of one or both of said endogenous BADC1 and BADC3 genes, and b) one or more transgene that alters metabolism of said target fatty acid.

In one embodiment, the present invention provides a progeny plant of a transgenic plant or part thereof that comprises: a) genomic mutation of fad2, fad3, fae1 or any combination thereof, b) reduced expression of one or both endogenous BADC1 and BADC3 genes, and c) one or more transgenes that alter metabolism of a target fatty acid.

The invention additionally provides a transgenic seed produced by any one or more of the methods the invention, wherein said transgenic seed comprises a transgenic plant cell having a) reduced or lacking expression of one or both of said endogenous BADC1 and BADC3 genes, and b) one or more transgene that alters metabolism of said target fatty acid. In one embodiment, said seed exhibits one or more of a) rescued or increased amount of total seed fatty acid per seed, b) rescued or increased amount of said target fatty acid per seed, and c) rescued or increased proportion of said target fatty acid relative to said total seed fatty acid per seed.

In one embodiment, the present invention provides a method of modifying a plant or part thereof for providing a transgenic seed, comprising producing a transgenic plant or part thereof that comprises a transgenic plant cell, said transgenic plant cell comprising a) reduced expression of one or both of endogenous BADC1 and BADC3 genes, and b) expression of one or more transgenes that alters metabolism of a target fatty acid. In one embodiment, said transgenic seed comprises a transgenic plant cell having a) reduced expression of one or both of said endogenous BADC1 and BADC3 genes, and b) one or more transgene that alters metabolism of said target fatty acid. In one embodiment, said seed exhibits one or more of a) a rescued or increased amount of total seed fatty acid per seed, b) rescued or increased amount of said target fatty acid per seed, and c) rescued or increased proportion of said target fatty acid relative to said total seed fatty acid per seed.

In one embodiment, the present invention provides a transgenic seed that produces a transgenic plant or part thereof that comprises: a) genomic mutation of fad2, fad3, fae1 or any combination thereof, b) reduced expression of one or both endogenous BADC1 and BADC3 genes, and c) one or more transgenes that alter metabolism of a target fatty acid.

The invention further provides a transgenic seed that produces the plant or part thereof of any one or more of the invention's methods, wherein said transgenic seed A) comprises a transgenic plant cell having a) reduced or lacking expression of one or both of said endogenous BADC1 and BADC3 genes, and b) one or more transgene that alters metabolism of said target fatty acid, and B) exhibits one or more phenotype of producing a plant with a) increased amount of total seed fatty acid per plant, b) improved establishment of one or both of roots and plant aerial parts, c) rescued or increased seed yield per plant, d) rescued or increased seed germination rate, e) rescued or increased amount of total seed fatty acid per seed, f) rescued or increased amount of said target fatty acid per seed, g) rescued or increased seed yield per plant, and h) rescued or increased proportion of said target fatty acid relative to said total seed fatty acid per seed.

In one embodiment, the present invention provides a transgenic seed that produces a transgenic plant or part thereof that comprises a transgenic plant cell, said transgenic plant cell comprising a) reduced expression of one or both of endogenous BADC1 and BADC3 genes, and b) expression of one or more transgenes that alters metabolism of a target fatty acid, wherein said transgenic seed A) comprises a transgenic plant cell having i) reduced expression of one or both of said endogenous BADC1 and BADC3 genes, and ii) one or more transgene that alters metabolism of said target fatty acid, and B) exhibits one or more phenotype of producing a plant with i) increased amount of total seed fatty acid per plant, ii) improved establishment of one or both of roots and plant aerial parts, iii) rescued or increased seed yield per plant, iv) rescued or increased seed germination rate, v) rescued or increased amount of total seed fatty acid per seed, vi) rescued or increased amount of said target fatty acid per seed, vii) rescued or increased seed yield per plant, and viii) rescued or increased proportion of said target fatty acid relative to said total seed fatty acid per seed.

The invention also provides a tissue culture of regenerable cells of any one or more of the transgenic plant or part thereof of the invention.

In one embodiment, the present invention provides a tissue culture of regenerable cells of a transgenic plant or part thereof that comprises a transgenic plant cell, wherein said transgenic plant cell comprises a) reduced or lacks expression of one or both of said endogenous BADC1 and BADC3 genes, and b) one or more transgene that alters metabolism of said target fatty acid, wherein said transgenic plant or plant part exhibits one or more phenotype of a) increased amount of total seed fatty acid per plant, b) improved establishment of one or both of roots and plant aerial parts, and c) rescued or increased seed yield per plant.

In one embodiment, the present invention provides a method of producing a target fatty acid using a transgenic plant or part thereof that comprises: a) genomic mutation of fad2, fad3, fae1 or any combination thereof, b) reduced expression of one or both endogenous BADC1 and BADC3 genes, and c) one or more transgenes that alter metabolism of said target fatty acid.

### BRIEF DESCRIPTION OF THE DRAWINGS

The file of this patent contains at least one drawing executed in color. Copies of this patent with color drawings will be provided by the Patent and Trademark Office upon request and payment of the necessary fee.

FIG. 1 Genotyping of badc1,3/fae1/FAH. Individual plants were genotyped to be fae1 homozygous via HinfI digested PCR fragment of FAE1 gene fragment; badc1 or badc3 homozygous were verified using PCR with the indicated gene-specific primer pairs and combinations with T-DNA-specific primer LBb1.

FIG. 2A-C Analysis of FIG. 2A BADC1, FIG. 2B BADC3 and FIG. 2C FAH gene expression in developing seeds. Transcript levels of BADC1, 3 were analyzed by qRT-PCR in 11- to 13-DAF developing seeds of fae1, fae1/FAH, badc1,3/fae1/FAH and badc1,3, n=3 biological replicates, and error bars represent SE. The relative expression levels are reported relative to the expression of the UBQ10 (At4g05320) transcript. Columns with different letters are significantly different (P<0.05) computed by the relative expression (REST) software algorithm using three biological replicates (Pfaffl et al., 2002).

FIG. 3A-3C Seed weight and FA content in seeds. (FIG. 3A) FA per seed. FA was determined by 5 pooled sets of 100 seeds each. (FIG. 3B) Oil content in seeds as a proportion of dry seed weight. Seed oil content, represented by total acyl lipids, was quantified by GC of fatty acid methyl esters. (FIG. 3C) Mean weight of transgenic seeds determined by five pooled sets of 100 seeds each. Error bars represent SE. Columns with different letters are significantly different using the Student's t test (P<0.05) and five biological replicates.

FIG. 4 Hydroxy FA content in seeds. HFA is expressed as a weight percentage of the total seed FA. Values represent the mean±standard deviation (n=3 pooled sets of 100 seeds). Student t test analysis found no significant difference between fae1/FAH and badc1,3/fae1/FAH (P<0.05).

FIG. 5 ACCase activity in developing seeds. [¹⁴C]Acetate incorporation into total lipids showed ACCase activity in 11- to 13-DAF developing seeds of fae1, fae1/FAH/badc1,3 and badc1,3. Specified different letters indicate significant differences (P<0.05) as determined by Student's t test using three biological replicates. Values are presented as means±SD of three biological replicates.

FIG. 6A-6C Seed germination and establishment. A total of 180 seeds in five equal replicates from each line were plated in ½ MS media containing 1% sucrose for 14 d. Germination is scored as seeds that produced a radicle, and seedlings that produced roots and green cotyledons were counted as being able to establish. The germination rates (FIG. 6A) and establishment rates (FIG. 6B) are calculated as to the percentage of total seeds plated. Values are presented as means±SD of five biological replicates. FIG. 6C Seed yield per plant. n=10, and error bars represent ±SE. Columns with different letters are significantly different (P<0.05) of the five replicates.

FIG. 7 EMS mutation caused truncation of FAE1 in fae1 mutant. FAE1 gene was amplified from fae1 mutant an its sequence showed a mutated codon (TGG1395TGA, highlighted in green).

FIG. 8 Analysis of fatty acid synthesis related gene expression in developing seeds. Transcript levels of genes were analyzed by qRT-PCR in 11- to 13-DAF developing seeds of fae1, fae1/FAH, badc1,3/fae1/FAH and badc1,3, n=3 biological replicates, and error bars represent SD. RT-qPCR values are presented as percentages of internal control normalized as described in “Materials and Methods.” No values were found to differ significantly (P<0.05) using three biological replicates computed by the relative expression (REST) software algorithm (Pfaffl et al., 2002).

FIG. 9 [¹⁴C]acetate incorporation assay in developing seeds of badc1,3. Developing seeds 11-13 days after flowering were collected from badc1,3 seeds and their fatty acid synthesis rates were determined by measuring the rate of [¹⁴C]acetate incorporation into FAs by total lipid extraction and scintillation counting. Incorporation of [¹⁴C]acetate between 20 and 100 minutes at a 20 minutes interval.

FIG. 10 Seed germination and establishment. Seeds from fae1, fae1/FAH, badc1,3/fae1/FAH and badc1,3 were germinated on ½ MS medium with 1% sugar plate and 7 and 10 day old plants were photographed.

FIG. 11 Hydroxy FA content in seeds. HFA is expressed as a weight percentage of the total seed FA. Values represent the mean±standard deviation (n=5 pooled sets of 100 seeds representing 5 biological replicates). Student t test analysis found no significant difference between CL37 and CL37/badc1 lines, but significant difference between CL37 and 4 CL37/badc3 lines (P<0.05).

FIG. 12A-12B Seed germination and establishment on media supplemented with sucrose. A total of 180 seeds in five replicates from each line were plated in ½ MS media supplemented with 1% sucrose. Germination is scored as seeds that produced a radicle, and seedlings that produced roots and green cotyledons were scored as establishment. The germination rates (FIG. 12A) and establishment rates (FIG. 12B) are calculated as to the percentage of total seeds plated. Values are presented as means±SD of five biological replicates. Genotypes with different letters are significantly different (P<0.05).

FIG. 13A-B Seed germination and establishment on media without sucrose. A total of 180 seeds in five replicates from each line were plated in ½ MS media. Germination is scored as seeds that produced a radicle, and seedlings that produced roots and green cotyledons were scored as being able to establish. The germination rates (FIG. 13A) and establishment rates (FIG. 13B) are calculated as to the percentage of total seeds plated. Values are presented as means±SD of five biological replicates. Genotypes with different letters are significantly different (P<0.05) of the five replicates.

FIG. 14 Seed production per plant. Plants of fae1, CL37, and CL37/badc3 lines 2 and 19 were grown side by side, and seeds were collected at maturity. Seed yields per plant were weighed. n=18, and error bars represent ±SE. Columns with different letters are significantly different (P<0.05).

FIG. 15 Vector diagram of FAH plant expression vector. RcFAH gene is placed under the control of seed-specific phaseolin promoter.

FIG. 16A: FAH (also referred to as RcFAH) nucleotide sequence: Gene ID #8267537, >NM_001323721.1 FAH mRNA.

FIG. 16B: FAH amino acid sequence: NP_001310650.1 oleate hydroxylase FAH12 [Ricinus communis].

FIG. 16C: Camelina BADC1, First isoform nucleotide sequence: >Csa04g042500.1.

FIG. 16D: Camelina BADC1, First isoform amino acid sequence >Csa04g042500.1.

FIG. 16E: Camelina BADC1, Second isoform nucleotide sequence >Csa06g030800.1.

FIG. 16F: Camelina BADC1, Second isoform amino acid sequence >Csa06g030800.1.

FIG. 16G: Camelina BADC1, Third isoform nucleotide sequence >Csa09g068300.1.

FIG. 16H: Camelina BADC1, Third isoform amino acid sequence >Csa09g068300.1.

FIG. 16I: Camelina BADC3, First isoform nucleotide sequence >Csa15g020290.1.

FIG. 16J: Camelina BADC3, First isoform amino acid sequence >Csa15g020290.1.

FIG. 16K: Camelina BADC3, Second isoform nucleotide sequence >Csa19g022480.1.

FIG. 16L: Camelina BADC3, Second isoform amino acid sequence >Csa19g022480.1.

FIG. 16M: Camelina BADC3, Third isoform nucleotide sequence >Csa01g018320.1.

FIG. 16N: Camelina BADC3, Third isoform amino acid sequence >Csa01g018320.1.

FIG. 16O: FAE1, AT4G34520, Coding sequence.

FIG. 16P: FAE1, AT4G34520, Protein Sequence.

FIG. 16Q: Arabidopsis FAD2, AT3G12120.1, Coding sequence.

FIG. 16R: Arabidopsis FAD2, AT3G12120.1, Protein Sequence.

FIG. 16S: Arabidopsis FAD3, AT2G29980.1, CDS.

FIG. 16T: Arabidopsis FAD3, AT2G29980.1, Protein.

FIG. 16U: mutant fatty acid elongation 1 (FAE1) DNA sequence (also see FIG. 7).

FIG. 16V: mutant fatty acid elongation 1 (fae1) protein sequence (also see FIG. 7).

FIG. 17A: E. coli Cyclopropane fatty acid synthase (EcCPS1) DNA, NCBI Gene ID: 944811; >NC_000913.3:1741413-1742561.

FIG. 17B: E. coli Cyclopropane fatty acid synthase (EcCPS1) protein, NP_416178.1.

FIG. 17C: Crepis palaestina delta 12 fatty acid epoxygenase GenBank #: Y16283.1; >Y16283.1:30-1154 Crepis palaestina mRNA for delta 12 fatty acid epoxygenase.

FIG. 17D: >CAA76156.1 delta 12 fatty acid epoxygenase [Crepis palaestina].

FIG. 17E: Crepis alpina delta-12 fatty acid acetylenase GenBank #: DQ289485.1; >DQ289485.1 Crepis alpina delta-12 fatty acid acetylenase (vFAD2) gene, complete cds

FIG. 17F: ABC00769.1 delta-12 fatty acid acetylenase [Crepis alpina].

FIG. 17G: Momordica charantia Conjugase (FadX) GenBank #: AF182521.1; >AF182521.1 Momordica charantia delta-12 oleic acid desaturase-like protein (FadX) mRNA, complete cds.

FIG. 17H: >AAF05916.1 delta-12 oleic acid desaturase-like protein [Momordica charantia].

### DEFINITIONS

“Wild-type” and “normal” are interchangeably used when in reference to any molecule or its level (e.g., amino acid sequence, and nucleic acid sequence, etc.) and/or phenomenon or its level (e.g., expression of a gene, transcription of a DNA sequence, translation of an mRNA molecule to an amino acid sequence) and/or phenotype or its level (e.g., seed yield per plant, amount of total seed fatty acid per seed, amount of a target fatty acid per seed, seed yield per plant, seed germination rate, proportion of a target fatty acid relative to total seed fatty acid per seed, amount of total seed fatty acid per plant, establishment of roots, establishment of plant aerial parts) to mean that the molecule or its level and/or phenomenon or its level and/or phenotype or its level is the same as found in nature without alteration by the hand of man (such as by chemical and/or molecular biological techniques, etc.).

“Expression” refers to the transcription and stable accumulation of sense or anti-sense RNA derived from a nucleic acid. “Expression” may also refer to translation of mRNA into a polypeptide or protein. As used herein, the term “antisense RNA” refers to an RNA transcript that is complementary to all or a part of a mRNA that is normally produced in a cell. The complementarity of an antisense RNA may be with any part of the specific gene transcript, i.e., at the 5′ non-coding sequence, 3′ non-translated sequence, introns, or the coding sequence. As used herein, the term “RNA transcript” refers to the product resulting from RNA polymerase-catalyzed transcription of a DNA sequence. When the RNA transcript is a perfect complimentary copy of the DNA sequence, it is referred to as the primary transcript or it may be an RNA sequence derived from post-transcriptional processing of the primary transcript and is referred to as the mature RNA.

“Reducing gene expression” and grammatical equivalents refers to a reduction in one or both of DNA transcription into mRNA, and mRNA translation into a protein molecule. In one embodiment, reducing gene transcription refers to the absence (or observable decrease) in the level of protein and/or mRNA product from the target gene. Specificity refers to the ability to inhibit the target gene without manifest effects on other genes of the cell and without any effects on any gene within the cell that is producing the dsRNA molecule. The inhibition of gene expression of a target gene as described herein may result in novel phenotypic traits in the plant. Reduced gene expression may be achieved by completely silencing or down-regulating expression of a gene and/or partial or incomplete silencing or down-regulation of a gene and/or introducing a mutation into the gene. Post-transcriptional gene suppression by anti-sense or sense-oriented RNA to regulate gene expression in plant cells is known in the art, as is the use of dsRNA to suppress genes in plants. Post-transcriptional gene suppression in plants may employ both sense-oriented and anti-sense-oriented, transcribed RNA that is stabilized, e.g., as a hairpin or stem-and-loop structure. In one embodiment, BADC genes are partially or totally silenced by expression of an RNAi cassette as described in WO 2017/039834 and WO 2018/009626.

“Mutation” for reducing gene expression includes deletion, insertion and/or substitution of one or more nucleotides of the gene or of sequences regulating expression of the gene. In one embodiment, said mutation comprises deleting at least a portion of the coding region, deleting the entire gene, deleting at least a portion of sequences that regulates transcription of the gene, introducing an insertion and/or a frameshift mutation, etc. so that at least one mutated allele contains a deletion of the translation start site, transcription start codon, at least a portion of the promoter region, at least a portion of the coding region, or any combination thereof. In one embodiment, said deleted sequences may be replaced with polynucleotides that are exogenous to the deleted gene sequences and that are flanked by sequences that are complementary to polynucleotide regions of the endogenous gene that flank the deleted gene sequences. In a further embodiment, at least one mutated allele is generated by site specific recombination, frame shift mutation, homologous recombination, CRISPR gene editing, or any combination thereof, in a cell such as an embryonic stem cell or germ cell.

“Genome editing” refers to the process of modifying (by insertion and/or deletion and/or substitution) of the nucleotide sequence of a genome sequence (e.g., coding sequence, non-coding sequence, tandem repeats, transposable elements, retrotransposons, long terminal repeats (LTRs), Non-long terminal repeats (Non-LTRs), etc.), preferably in a pre-determined targeted manner. In some embodiments, genome editing methods are exemplified by the CRISPR-endonuclease system, which produces a site-specific modification of a target DNA as described in Doudna et al., U.S. Pat. No. 10,000,772 (incorporated by reference). “CRISPR” (“clusters of regularly interspaced short palindromic repeats”) gene editing is exemplified in Examples 9 and 10, and is used to knockout plant genes in melon (Hooghvorst et al. (2019) Scientific Reports 9:17077) and Brassica napus FAD2 (Okuzaki et al. (2018) Plant Physiology and Biochemistry, Volume 131, October 2018, Pages 63-69).

“Transformation” is a process of introducing a DNA sequence or construct (e.g., a vector or expression cassette) into a cell or protoplast in which that exogenous DNA is incorporated into a chromosome or is capable of autonomous replication.

“Stable transformation” of a cell with a transgene means that the transgene is integrated within the cell's genome. Methods for genetic transformation of plants (including use of regulatory elements, terminators, marker genes) and for production and characterization of stably transformed plants are known in the art (WO 2018/009626).

“Transgenic” and “genetically engineered” cell refer to a cell whose genome has been manipulated by any molecular biological technique, including, for example, the introduction of a transgene, homologous recombination, knockin of a gene, knockout of a gene, and/or CRISPR gene editing.

The term “transgene” refers to any nucleic acid sequence that is introduced into the cell by experimental manipulations. A transgene may be an “endogenous” DNA sequence or a “heterologous DNA sequence.”

“Endogenous” molecule (such as nucleotide sequence, amino acid sequence, fatty acid) is a molecule natively found in nature in a host cell or a cell of the same species. In one embodiment, an endogenous sequence may be overexpressed or expressed at a higher level compared to wildtype and still be considered endogenous.

“Heterologous” and “foreign” molecule (such as nucleotide sequence, amino acid sequence, fatty acid) is a molecule that is not endogenous. In one embodiment, a heterologous sequence contains some modification (e.g., mutation, the presence of a selectable marker gene, etc.) relative to the naturally-occurring sequence. In this respect, the heterologous sequence may be native to the host genome, but be rearranged with respect to other genetic sequences within the host sequence. For example, a regulatory sequence may be heterologous in that it is linked to a different coding sequence relative to the native regulatory sequence. In addition, a particular sequence can be “heterologous” with respect to a cell or organism into which it is introduced (for example, a sequence that does not naturally occur in that particular cell or organism).

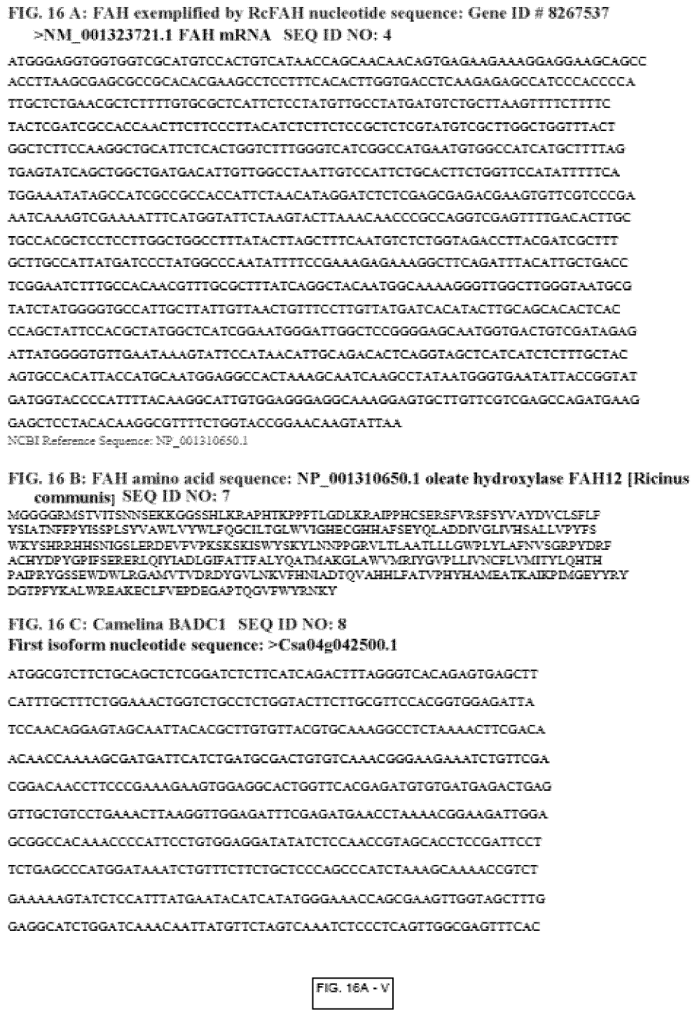

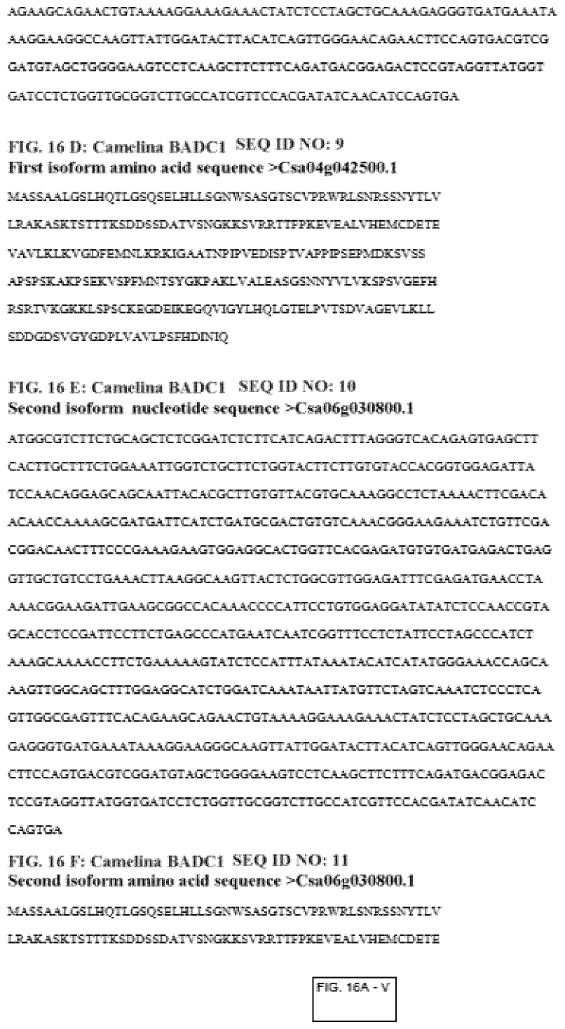

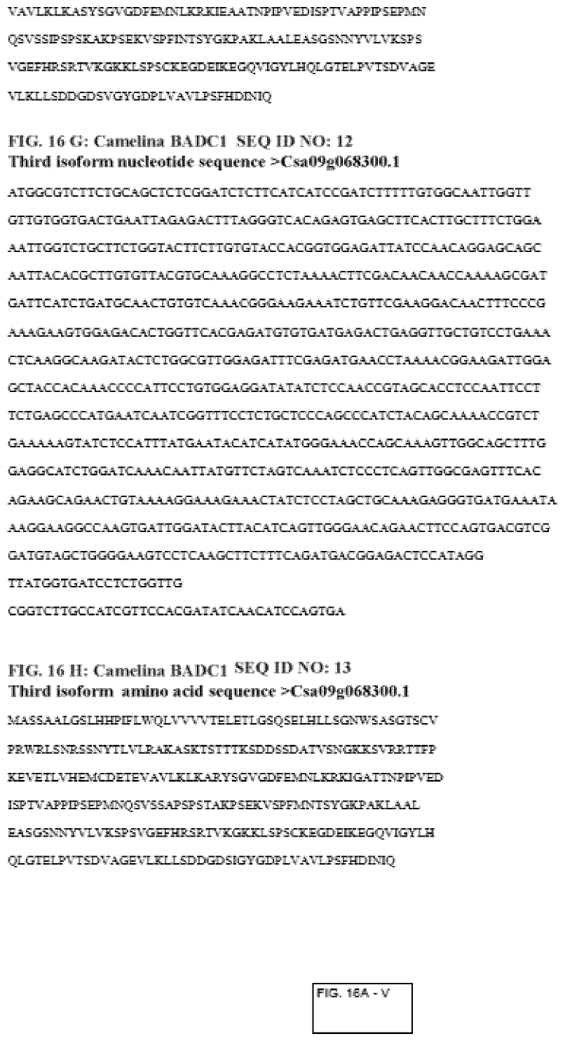

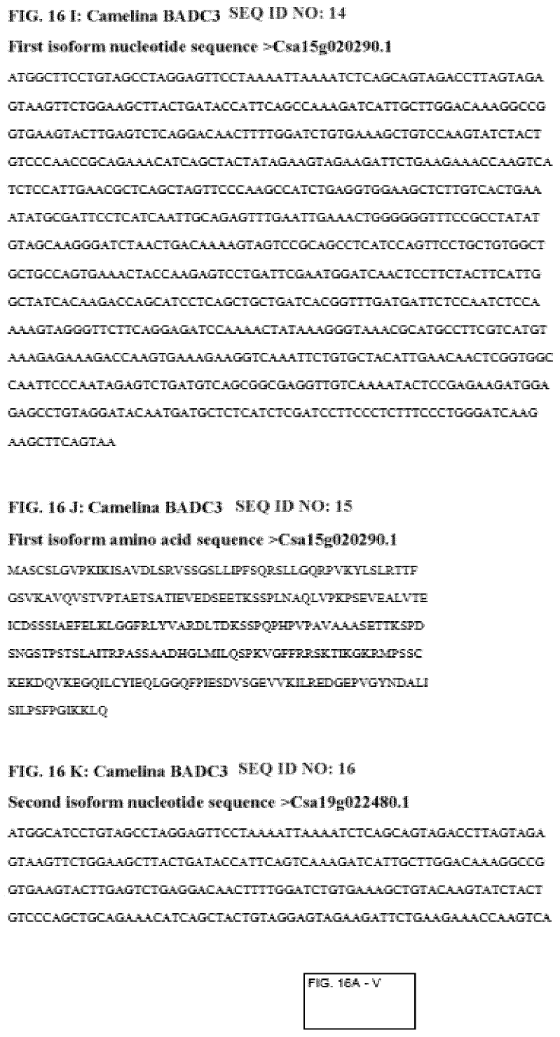

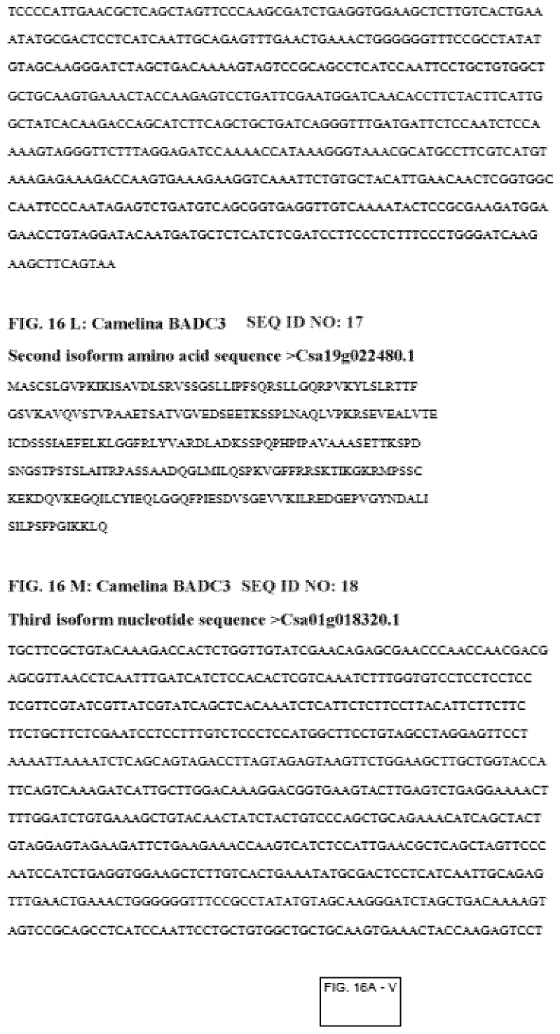

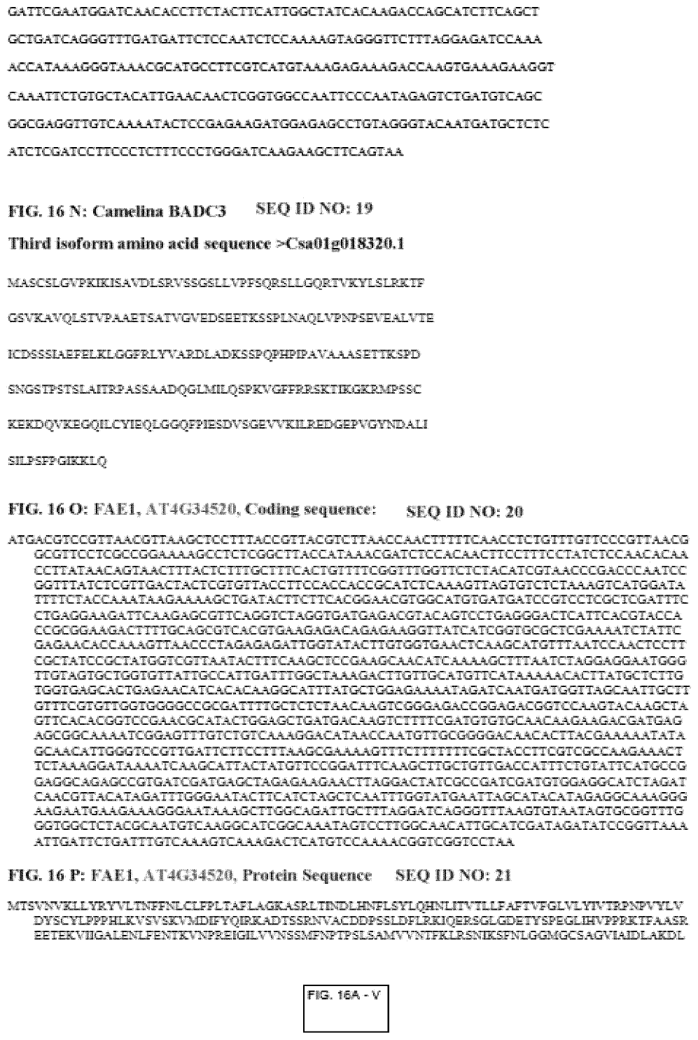

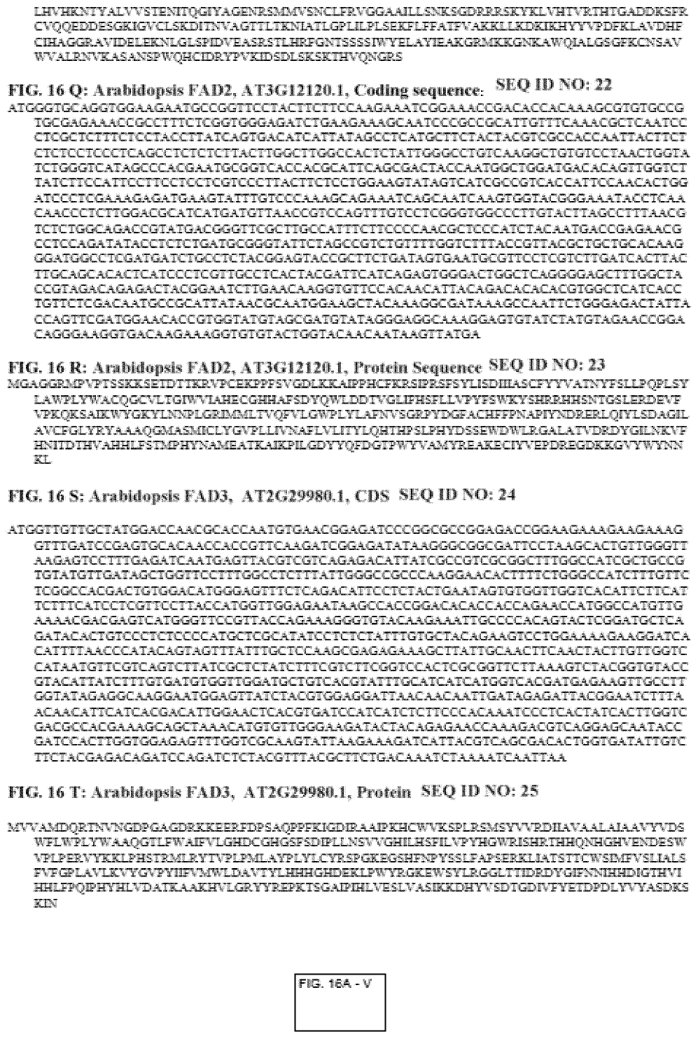

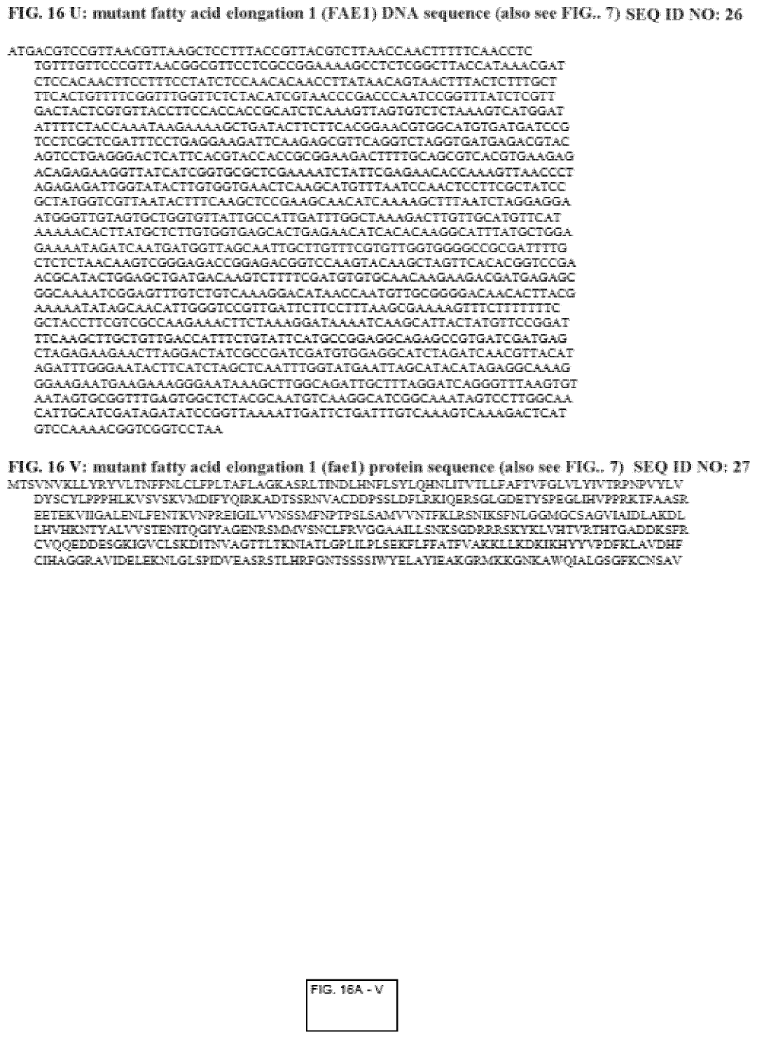

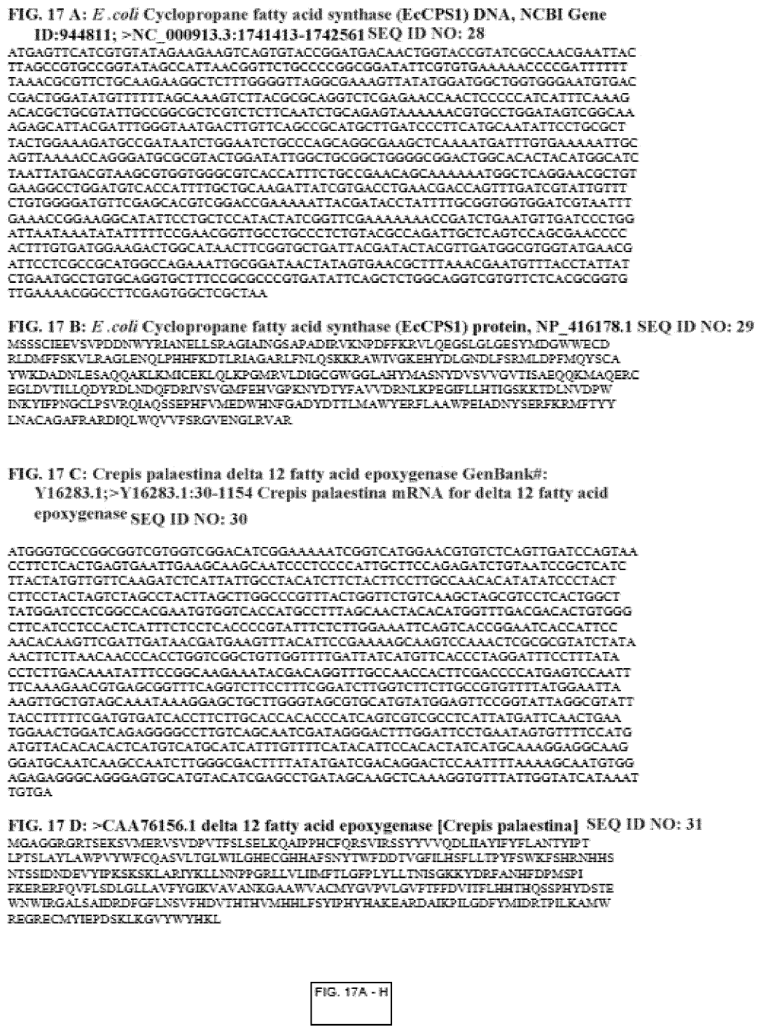

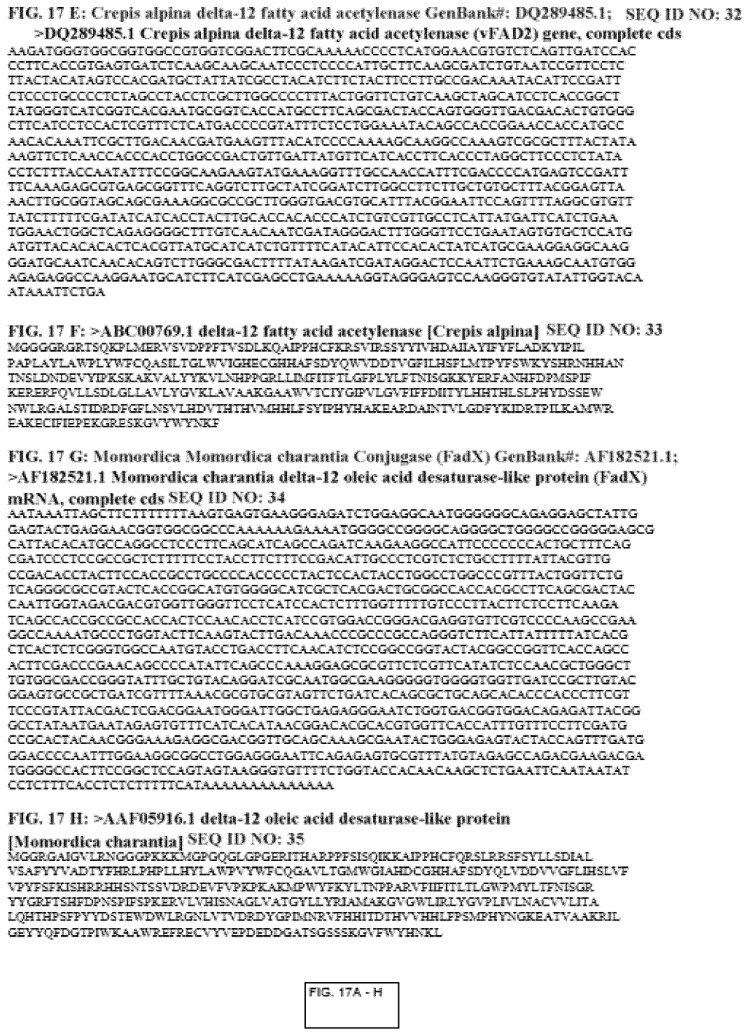

“BADC1” gene refers to accession AT3G56130 and/or orthologs thereof. In one embodiment, BADC1 gene is exemplified by one or more of the three isoform sequences in FIGS. 16C, 16E and 16G, including nucleotide sequences that comprise from about 34%, 40%, 50%, 60%, 62%, 70%, 80%, 85%, 90%, 95% to about 100% sequence identity to sequences in FIGS. 16C, 16E and 16G, or a complement thereof. In another embodiment, BADC1 gene encodes a polypeptide comprising from about 34%, 40%, 50%, 60%, 62%, 70%, 80%, 85%, 90%, 95% to about 100% sequence identity to any one of the three isoform polypeptide sequences in FIGS. 16D, 16F and 16H. In one embodiment, BADC1 gene is exemplified by sequences described in WO 2018/009626, including nucleotide sequences that comprise from about 34%, 40%, 50%, 60%, 62%, 70%, 80%, 85%, 90%, 95% to about 100% sequence identity to WO 2018/009626's nucleotide sequence SEQ ID NO: 2, or a complement thereof. In another embodiment, BADC1 gene encodes a polypeptide comprising from about 34%, 40%, 50%, 60%, 62%, 70%, 80%, 85%, 90%, 95% to about 100% sequence identity to WO 2018/009626's polypeptide sequence SEQ ID NO: 1.

“BADC3” gene refers to accession AT3G15690 and/or orthologs thereof. In one embodiment, BADC3 gene is exemplified by one or more of the three isoform sequences in FIGS. 16I, 16K and 16M, including nucleotide sequences that comprise from about 34%, 40%, 50%, 60%, 62%, 70%, 80%, 85%, 90%, 95% to about 100% sequence identity to sequences in FIGS. 16I, 16K and 16M, or a complement thereof. In another embodiment, BADC3 gene encodes a polypeptide comprising from about 34%, 40%, 50%, 60%, 62%, 70%, 80%, 85%, 90%, 95% to about 100% sequence identity to any one of the three isoform polypeptide sequences in FIGS. 16J, 16L and 16N. In one embodiment, BADC3 gene is exemplified by sequences described in WO 2018/009626, including nucleotide sequences that comprise from about 34%, 40%, 50%, 60%, 62%, 70%, 80%, 85%, 90%, 95% to about 100% sequence identity to WO 2018/009626's nucleotide sequence SEQ ID NO: 6, or a complement thereof. In another embodiment, BADC1 gene encodes a polypeptide comprising from about 34%, 40%, 50%, 60%, 62%, 70%, 80%, 85%, 90%, 95% to about 100% sequence identity to WO 2018/009626's polypeptide sequence SEQ ID NO: 5.

“Ortholog” genes refers to genes that are related by vertical descent from a common ancestor and encode proteins with the same function in different species. In one embodiment, ortholog nucleotide sequences comprise from about 34%, 40%, 50%, 60%, 62%, 70%, 80%, 85%, 90%, 95% to about 100% sequence identity. In another embodiment, ortholog polypeptide sequences comprise from about 34%, 40%, 50%, 60%, 62%, 70%, 80%, 85%, 90%, 95% to about 100% sequence identity. By contrast, “paralogs” are homologous genes that have evolved by duplication and code for protein with similar, but not identical functions. Exemplary orthologs of BADC1 gene and BADC3 gene, and proteins encoded by these genes, are described in WO 2018/009626, and the following Table 1 with respect to Camelina sativa, Glycine max and Brassica napus.

TABLE 1
BADC orthologs

| A. thaliana | | sub-genome/ | | |
|---|---|---|---|---|
| gene | TAIR ID | block¹ | Ensembl Plants gene ID | species |
| BADC1 | AT3G56130 | LF | Csa04g042500 | Camelina sativa |
| BADC1 | AT3G56130 | MF1 | Csa06g030800 | Camelina sativa |
| BADC1 | AT3G56130 | MF2 | Csa09g068300 | Camelina sativa |
| BADC1 | AT3G56130 | A LF | GSBRNA2T00037117001 | Brassica napus |
| BADC1 | AT3G56130 | C LF | GSBRN A2T00155998001 | Brassica napus |
| BADC1 | AT3G56130 | n/a | GLYMA_11G35740 | Glycine max |
| BADC1 | AT3G56130 | n/a | GLYMA_18G02670 | Glycine max |
| BADC2 | AT1G52670 | LF | Csa17g092720 | Camelina sativa |
| BADC2 | AT1G52670 | MF1 | Csa14g061920 | Camelina sativa |
| BADC2 | AT1G52670 | MF2 | Csa03g059640 | Camelina sativa |
| BADC3 | AT3G15690 | LF | Csa15g020290 | Camelina sativa |
| BADC3 | AT3G15690 | MF1 | Csa19g022480 | Camelina sativa |
| BADC3 | AT3G15690 | MF2 | Csa01g018320 | Camelina sativa |
| BADC3 | AT3G15690 | A MF1 | GSBRNA2T00010009001 | Brassica napus |
| BADC3 | AT3G15690 | A LF | GSBRNA2T00104942001 | Brassica napus |
| BADC3 | AT3G15690 | C MF1 | GSBRNA2T00018165001 | Brassica napus |
| BADC3 | AT3G15690 | C LF | GSBRNA2T00044654001 | Brassica napus |
| BADC2/3² | AT1G52670/ | n/a | GLYMA_13G44040 | Glycine max |
| | AT3G15690 | | | |
| BADC2/3 | AT1G52670/ | n/a | GLYMA_15G01300 | Glycine max |
| | AT3G15690 | | | |

In Table 1, sub-genome information is only given for Brassicaceae species. BADC2 and BADC3 are thought to be derived through a gene duplication event at the base of the Brassicaceae. Therefore, GLYMA13G44040 and GLYMA15G01300 are homologs to an ancient precursor of BADC2 and BADC3.

“Seed-specific” promoter refers to a promoter that preferentially controls expression of an operably linked transgenes in seed products. Seed specific promoters are exemplified by the seed-specific phaseolin promoter, Napin promoter, β-conglycinin promoter, pea legumin legA promoter, and foxtail millet pF128 promoter.

“Fatty acid” refers to a carboxylic acid consisting of a hydrocarbon chain and a terminal carboxyl group, especially any of those occurring as esters in fats and oils. Fatty acid includes “unusual fatty acid,” “special fatty acid” and “specialty fatty acid,” which interchangeably refer to any fatty acid that is naturally found in a plant or plant part (such as seed) at less than 2 mole percent. Unusual fatty acids in seed oils from different species have identified more than 200 naturally occurring fatty acids of which 18 representatives are listed in the following Table 2 (David Hildebrand, “Production of Unusual Fatty Acids in Plants—AOCS Lipid Library” 2018):

TABLE 2

| Common name | Chemical name | High accumulator | % UFA |
|---|---|---|---|
| alchornoic | 14-epoxy,cis-11-eicosenoic | Alchornea cordifolia | 50 |
| axillarenic | 11,13-dihydroxy-tetracos-trans-9-enoic | Baliospermum axillare | 3 |
| calendic | trans-8,trans-10,cis-12-octadecatrienoic | Calendula officinalis | 63 |
| catalpic | trans-9,trans-11,cis-13-octadecatrienoic | Catalpa bignonioides | |
| dimorphecolic | 9-hydroxy-trans,10-trans-12-octadecadienoic | Dimorphotheca | 60 |
| | | pluvialis | |
| coronaric | 9-epoxy,cis-12-octadecenoic | Chrysanthemum | |
| | | coronarium | |
| crepenynic | octadec-cis-9-en-12-ynoic | Crepis alpina | 74 |
| eleostearic | cis-9,trans-11,trans-13-octadecatrienoic | Aleurites fordii | 80 |
| epoxystearic | 9-epoxy-octadecanoic | Tragopogon | 3 |
| | | porrifolius | |
| isanolic | 8-hydroxy-octadec-17-en-9,11-diynoic | Ongokea gore | |
| isoricinoleic | 9-hydroxy-12-cis-octadecenoic | Wrightia coccinea | 76 |
| licanic | 4-oxo-cis-9,trans-11,trans-13-octadecatrienoic | Licania rigida | 78 |
| lesquerolic | 14-hydroxy-cis-11-eicosenoic | Lesquerella fendleri | 55 |
| parinaric | cis-9,trans-11,trans-13,cis-15- | Parinarium laurunum | 54 |
| | octadecatetraenoic | | |
| punicic | cis-9,trans-11,cis-13-octadecatrienoic | Punicia granatum | 86 |
| phloionolic | 9,10,18-trihydroxy octadecanoic | Chamaepeuce afra | 9 |
| ricinoleic | 12-hydroxy-9-cis-octadecenoic | Ricinus communis | 88 |
| vernolic | 12-epoxy,cis-9-octadecenoic | Vernonia galamensis | 80 |

In one embodiment, the fatty acid comprises one or more of hydroxyl fatty acids, medium-chain fatty acids, very-long-chain fatty acids (VLCFAs), monounsaturated fatty acids (MUFAs), γ-linolenic acid, stearidonic acids, α-eleostearic acid, conjugated fatty acids, epoxy fatty acids, cyclic fatty acids and acetylenic fatty acids, medium-chain fatty acids such as lauric acid and derivatives; very-long-chain fatty acids (VLCFAs) such as erucic acid; monounsaturated fatty acids (MUFAs) such as palmitoleic acid (also referred to as cis-9-hexadecenoic acid (16:1Δ9)), oleic acid (18:1Δ9) and petroselinic acid (18:1Δ6); γ-Linolenic acid (Δ6,9,12-18:3); stearidonic acids such as octadecatetraenoic acid (Δ6,9,12,15-18:4); conjugated fatty acids such as α-Eleostearic acid (9-cis,11-trans,13-trans-octadecatrienoic acid), calendic acid (trans-8,trans-10,cis-12-octadecatrienoic acid), punicic acid (cis-9,trans-11,cis-13-octadecatrienoic acid, parinaric acid (cis-9,trans-11,trans-13,cis-15-octadecatetraenoic acid), licanic acid (4-oxo-cis-9,trans-11,trans-13-octadecatrienoic acid), and catalpic acid (trans-9,trans-11,cis-13-octadecatrienoic acid); epoxy fatty acids, such as vemolic (cis-12,13-epoxyoctadeca-cis-9-enoic) and coronaric (cis-9,10-epoxyoctadeca-cis-12-enoic) acids, acetylenic fatty acids, 9,10-epoxystearic acid, alchornoic acid (14,15-epoxycis-11-eicosenoic acid), and 15-epoxy-cis-9,cis-12-octadecadienoic acid; and acetylenic fatty acids such as crepenynic acid (octadec-9-en-12-ynoic acid) (David Hildebrand, “Production of Unusual Fatty Acids in Plants—AOCS Lipid Library” 2018). Examples of hydroxyl fatty acids include ricinoleic acid (A-12-hydroxy-9-cis-octadecanoic acid or 12-d-hydroxy-octadeca-cis-9-enoic acid) and densipoleic acid (9Z,12R,15Z)-12-hydroxyoctadeca-9,15-dienoate.

“Rescued” means that if a first plant exhibits a first phenotype that is altered (increased or decreased) by a first mutation to a first nucleotide or polypeptide sequence, then a second mutation to the same or different nucleotide or polypeptide sequence is said to “rescue” the first phenotype if a second plant that has both the first and second nucleotide or polypeptide mutations exhibits substantially the same phenotype as the first plant, and the phenotype is said to be “rescued” by the second mutation. For example, if a wild type plant exhibits a first level of seed germination that is decreased by overexpression of FAH gene, then a mutation to genomic BADC3 gene is said to rescue the seed germination phenotype if a second plant that has both the FAH gene and mutant genomic BADC3 gene exhibits substantially the same level of seed germination as the wild type plant, and the seed germination phenotype is said to be “rescued” by the BADC3 gene mutation.

“Seed yield” means the number of seeds and/or weight of seeds.

“Germination” refers to the process whereby the seed coat splits and root and cotyledons start to poke out of the seed, after a dry seed is exposed to desired germination conditions such as water, light, soil, etc.

“Establishment” refers to the process whereby roots and aerial parts of a plant start to grow as a seedling starts to develop after a dry seed is exposed to desired germination conditions such as water, light, soil, etc. “Improved establishment” of roots or aerial parts of the plant refers to an increase in one or more of the length, girth and branching or roots and/or aerial parts of the plant.

“Plant” refers to a living thing that grows in the earth and has a stem, leaves, and roots, exemplified by organisms that contain orthologs to the Arabidopsis thaliana BADC genes, such as Amborella trichopoda, Arabidopsis lyrata, Arabidopsis alpine, Arachis hypogaea, Auxenochlorella protothecoides, Brassica napus, Brassica rapa, Camelina sativa, Capsella rubella, Cathamus tinctorius, Chlamydomonas reinhardtii, Chlorella variabilis, Cicer arietinum, Citrus clementina, Citrus sinensis, Coccomyxa subellipsoideas C-169, Coffea canephora, Cucumis melo, Cucumis sativus, Elaeis guineensis, Erythranthe guttata, Eucalyptus grandis, Eutrema salsugineum, Fragaria vesca, Genlisea aurea, Glycine max, Helianthus annuus, Helicosporidium ATCC 50920, Jatropha curcas, Lotus japonicas, Medicago truncatula, Marus notabilis, Musa acuminate, Nelumbo nuciera, Nicotiana sylvestris, Nicotiana tomentosiformis, Phaseolus vulgaris, Pheonix dactylifera, Physcomitrella patens, Picea sitchensis, Polytomella parva, Populus trichocarpa, Prunus mume, Prunes persica, Pyrus x bretschneideri, Ricinus communis, Selaginella moellendorffli, Sesamum indicum, Solanum lycopersicum, Solanum tuberosum, Theobroma cacao, Thlaspi arvense, Vitis viniera, or Volvox carteri.

A cell or organism is “homozygous” for a particular gene when identical alleles of the gene are present on all the homologous chromosomes. Thus, a diploid cell is homozygous for a particular gene when the cell contains two identical alleles of the gene. A cell or organism is “homozygous null” (also referred to as “nullizygous” and “nullizygote”) for a particular gene when it contains only mutant alleles for the same gene, and all the mutant alleles are complete loss-of-function (i.e., “null”) alleles. Thus, a diploid cell is homozygous null for a particular gene when the cell contains two null alleles of the gene. Null mutant BADC (i.e., BADC1 and/or BADC3) plants may be generated by crossing a male transgenic plant and a female transgenic plant each bearing one artificially mutated BADC allele in its germ cells.

A cell or organism is “heterozygous” for a particular gene when different alleles of the gene are present on the homologous chromosomes. Thus, a diploid cell is heterozygous for a particular gene when the cell contains two different alleles (e.g., one wild-type allele and one mutant allele) of the gene.

“Breeding” and “crossing” and “crossbreeding” interchangeably refers to the process of selectively propagating plants with desirable characteristics using closely or distantly related individuals to produce new plant varieties or lines with desirable properties. In one embodiment, crossing a plant line having one or more transgenes and/or genomic modifications relative to a starting plant line means the techniques that result in the one or more transgenes and/or genomic modifications of the invention being introduced into a plant line by crossing a plant of a starting line with a plant of a donor plant line that comprises one or more transgenes and/or genomic modifications of the invention. Methods for breeding (such as to produce plants that are homozygous for a transgene) are disclosed herein and known in the art (WO 2018/009626).

Plant “part” refers to a plant cell and/or tissue and/or organ, exemplified by seed, leaf, pollen, ovule, fruit, rootstock, flower and scion. In one embodiment, the plant tissue comprises tissue obtained directly or indirectly (e.g., by tissue culture of regenerable cells) from the plant. In a further embodiment, the plant part comprises a seed that produces, and/or is produced by, a plant produced by the presently disclosed methods.

“Regenerable” plant cells include protoplasts and embryogenic cells. Illustrative methods for tissue culture for the regeneration of cereals from protoplasts have been described (Toriyama et al., 1986; Yamada et al., 1986; Abdullah et al., 1986; Omirulleh et al., 1993 and U.S. Pat. No. 5,508,184; each specifically incorporated herein by reference in its entirety).

“Progeny” denotes the offspring of any generation of a parent plant prepared in accordance with the instant invention. In one embodiment, the progeny exhibits one or more phenotypes of the parent plant, and comprises one or more of the transgenes and one or more of the genomic modifications of the parent plant.

The terms “reduce,” “inhibit,” “diminish,” “suppress,” “decrease,” and grammatical equivalents (including “lower,” “smaller,” etc.) when in reference to the level of any molecule (e.g., amino acid sequence, and nucleic acid sequence, etc.) and/or phenomenon (e.g., level of expression of a gene, level of transcription of a DNA sequence, level of translation of an mRNA molecule to an amino acid sequence) and/or phenotype (e.g., seed yield per plant, amount of total seed fatty acid per seed, amount of a target fatty acid per seed, seed yield per plant, seed germination rate, proportion of a target fatty acid relative to total seed fatty acid per seed, amount of total seed fatty acid per plant, establishment of roots, establishment of plant aerial parts) in a first composition (e.g., first plant cell) relative to a second composition (e.g., second plant cell), mean that the quantity of molecule and/or phenomenon and/or phenotype in the first composition is lower than in the second composition by any amount that is statistically significant using any art-accepted statistical method of analysis. In one embodiment, the quantity of molecule and/or phenomenon and/or phenotype in the first composition is at least 10% lower than, at least 25% lower than, at least 50% lower than, at least 75% lower than, at least 90% lower and/or 100% lower than the quantity of the same molecule and/or phenomenon and/or phenotype in the second composition. In one embodiment, the first composition lacks (i.e., contains 0% of) the molecule and/or phenomenon and/or phenotype.

The terms “increase,” “elevate,” “raise,” and grammatical equivalents (including “higher,” “greater,” etc.) when in reference to the level of any molecule (e.g., amino acid sequence, and nucleic acid sequence, etc.) and/or phenomenon (e.g., level of expression of a gene, level of transcription of a DNA sequence, level of translation of an mRNA molecule to an amino acid sequence) and/or phenotype (e.g., seed yield per plant, amount of total seed fatty acid per seed, amount of a target fatty acid per seed, seed yield per plant, seed germination rate, proportion of a target fatty acid relative to total seed fatty acid per seed, amount of total seed fatty acid per plant, establishment of roots, establishment of plant aerial parts) in a first composition (e.g., first plant cell) relative to a second composition (e.g., second plant cell), mean that the quantity of molecule and/or phenomenon and/or phenotype in the first composition is higher than in the second composition by any amount that is statistically significant using any art-accepted statistical method of analysis. This includes, without limitation, a quantity of molecule and/or phenomenon and/or phenotype in the first composition that is at least 10% greater than, at least 15% greater than, at least 20% greater than, at least 25% greater than, at least 30% greater than, at least 35% greater than, at least 40% greater than, at least 45% greater than, at least 50% greater than, at least 55% greater than, at least 60% greater than, at least 65% greater than, at least 70% greater than, at least 75% greater than, at least 80% greater than, at least 85% greater than, at least 90% greater than, and/or at least 95% greater than the quantity of the same molecule and/or phenomenon and/or phenotype in the second composition.

The terms “alter” and “modify” when in reference to the level of any molecule (e.g., amino acid sequence, and nucleic acid sequence, etc.) and/or phenomenon (e.g., level of expression of a gene, level of transcription of a DNA sequence, level of translation of an mRNA molecule to an amino acid sequence) and/or phenotype (e.g., seed yield per plant, amount of total seed fatty acid per seed, amount of a target fatty acid per seed, seed yield per plant, seed germination rate, proportion of a target fatty acid relative to total seed fatty acid per seed, amount of total seed fatty acid per plant, establishment of roots, establishment of plant aerial parts) in a first composition (e.g., first plant cell) relative to a second composition (e.g., second plant cell), mean that the quantity of molecule and/or phenomenon and/or phenotype in the first composition refer to an increase and/or decrease in the level of molecule and/or phenomenon and/or phenotype.

### DESCRIPTION OF THE INVENTION

Hundreds of naturally occurring specialized fatty acids (FA) may have potential as chemical feedstocks if they can be produced at large scale by crop plants. However, transgenic expression of their biosynthetic genes has generally been accompanied by undesirable reductions in oil yield. For example, expression of Ricinus fatty acid hydroxylase (FAH) in the Arabidopsis fatty acid elongation mutant fae1 resulted in a 50% reduction of FA synthesis rate that was attributed to inhibition of acetyl Co-A carboxylase (ACCase) by an undefined mechanism. The hypothesis that the ricinoleic acid-dependent decrease in ACCase activity is mediated by biotin attachment domain-containing (BADC) proteins was tested.

BADCs are inactive homologs of biotin carboxy carrier protein that lack a biotin cofactor and can inhibit ACCase. Arabidopsis contains three BADC genes. To reduce expression levels of BADC1 and BADC3 in fae1/FAH, homozygous badc1,3/fae1/FAH was created. The rate of FA synthesis in badc1, 3/fae1/FAH seeds doubled relative to fae1/FAH, restoring it to fae1 levels, increasing both native FA and HFA accumulation. Total FA per seed, seed oil content and seed yield per plant all increased in badc1,3/fae1/FAH, to 5.8 μg, 37% and 162 mg, respectively, relative to 4.9 μg, 33% and 126 mg, respectively, for fae1/FAH. Transcript levels of fatty acid synthesis-related genes including ACCase subunits did not significantly differ between badc1,3/fae1/FAH and fae1/FAH. These results demonstrate that BADC1 and BADC3 mediate ricinoleic acid-dependent inhibition of FA synthesis. It is proposed that BADC-mediated FAS (fatty acid synthesis) inhibition may be a general mechanism that limits FA accumulation in specialized FA-accumulating seeds.

A longstanding crop improvement goal has been to exploit knowledge of specialized fatty acid synthesis from plants and microbes by reconstructing their synthetic pathways in crop production plants (Napier, 2007). If successful, this would allow the production of chiral fatty acid feedstocks in an inexpensive and scalable manner. However, a barrier to progress in this area was the discovery that upon the accumulation of specialized fatty acids seed oil yields are significantly decreased (Cahoon et al., 2007; Haslam et al., 2013; Vanhercke et al., 2013; Bates et al., 2014). An example of this comes from attempts to increase the accumulation of hydroxy fatty acid (HFA) in seed oils, of which much of the work has been performed in the model system Arabidopsis (Lu et al., 2006).

HFAs contain one or more hydroxy group(s) on a fatty acid backbone, which confer beneficial properties such as higher viscosity and chemical reactivity. The hydroxyl group of HFAs make them useful chemical feedstocks for the production of a wide range of industrial products including but not limited to: resins, waxes, nylons, plastics, lubricants, cosmetics, and additives for coatings and paints (Kim et al., 2000). Moreover, HFAs could be used as intermediates in the production of biodegradable plastics, cyclic lactones and pharmaceuticals (Wang et al., 2012). Industrial use of HFAs are available from natural sources such as castor beans which may limit their availability. Isolation of the oleate hydroxylase FAH from castor bean over two decades ago raised the possibility of ricinoleic acid production in high-yielding oilcrops (van de Loo et al., 1995). However, in contrast to castor beans that accumulate approximately 90% of its FA as ricinoleic acid, transgenic Arabidopsis fatty acid elongation1 (fae1) mutant expressing the FAH i.e., fae1/FAH, (a line designated CL37) accumulated only 17% HFA in its total seed oil (Lu et al., 2006). The seeds of fae1/FAH also displayed many physiological deficits including reduced oil content and seed weight, low seed yield per plant compared with its parental fae1 line, and seed germination was also delayed (Adhikari et al., 2016).

Investigation of the reduced oil content of fae1/FAH revealed its FA synthesis rate was reduced compared to the parental fae1 line (Bates et al., 2014). While the molecular basis for this reduction in FA synthesis has not been reported, several attempts at overcoming it have proved at least partially successful. For example, overexpressing a master transcriptional regulator of fatty acid synthesis WRINKLED1 (Adhikari et al., 2016) or a lipid droplet associated factor SEIPIN1 to increase lipid droplet size (Lunn et al., 2018). Development defects of HFA-accumulating seeds are partially mitigated upon the expression of several castor acyltransferases (Lunn et al., 2018). Stacking the expression of several castor acyltransferases, including GPAT9, LPAT2, and PDAT1A along with the castor hydroxylase fae1/FAH seeds produced abundant tri-HFA TAG, restored seed oil content and partially restored seedling establishment (Lunn et al., 2019). The expression of phosphatidylcholine:diacylglycerol cholinephosphotransferase (PDCT), encoded by the REDUCED OLEATE DESATURATION1 (ROD1) gene (Lu et al., 2009) which channels about 40% of the flux of polyunsaturated fatty from PC into DAG for TAG synthesis was found to potentiate efficient accumulation of HFA in Arabidopsis (Hu et al., 2012).

In dicotyledonous plants, heteromeric acetyl-CoA carboxylase (ACCase) catalyzes the first committed step of de novo fatty acid biosynthesis. This enzyme complex consists of four catalytic subunits: biotin carboxylase (BC), carboxyltransferase (CT)-α, CT-β, and biotin carboxyl carrier protein (BCCP) (Salie et al., 2016). The two BCCP isoforms (BCCP1 and BCCP2) of Arabidopsis ACCase can interact with Biotin/lipoyl attachment domain containing (BADC) proteins (Feria Bourrellier et al., 2010). BADCs are BCCP homologs that contain a biotin attachment motif, but critically lack a biotinylation site. BADC proteins can act as negative regulators of ACCase due to their lack of the biotin adduct required for carboxylation (Salie et al., 2016) and a role for them in ACCase assembly was recently proposed. These proteins have been reported to significantly inhibit ACCase activity in both E. coli and Arabidopsis (Salie et al., 2016), and it was recently proposed that they can sense pH changes (Ye et al., 2020). An additional role for BADCs in ACCase assembly has also been proposed (Shivaiah et al., 2020).

Three BADC genes have been identified in Arabidopsis, single badc1, badc2, badc3 Arabidopsis knock-out mutants do not exhibit significant changes in oil content relative to wild type plants (Keereetaweep et al., 2018), while the badc1badc3 (badc1,3) double mutant showed increased fatty acid synthesis rate and a remarkable 25% increase in seed oil content (Keereetaweep et al., 2018).

In this context, badc1,3/fae1/FAH homozygous plant were generated by crossing badc1,3 double mutant with CL37, an Arabidopsis fae1 line expressing FAH (Lu et al., 2006). Downregulation of BADC1 and BADC3 in fae1/FAH doubled the rate of FA synthesis in developing seeds, restoring it to fae1 levels, and increased both native FA and HFA accumulation.

### DISCUSSION OF EXEMPLARY EMBODIMENTS

It was previously reported that the accumulation of HFA in Arabidopsis seeds resulted in feedback inhibition of FA synthesis (Bates et al., 2014), with ACCase activity reduced by approximately 50% relative to the parental fae1 line. ACCase is often considered a rate limiting enzyme for FA synthesis and is therefore under tight genetic and biochemical regulation by a variety of mechanisms (Salie et al., 2016; Ye et al., 2020). In this study, we investigated the effects of null mutations in two negative regulatory subunits of ACCase i.e., badc1 and badc3 in FAH-expressing Arabidopsis seeds with respect to FA synthesis, common FA and HFA accumulation. The data demonstrates that eliminating BADC1 and BADC3 alleviates the HFA-dependent feedback inhibition of ACCase that results in a doubling FAS rate in badc1,3/fae1/FAH seeds restoring them to that of the parental fae1 line. Seed FA content of badc1,3/fae1/FAH was also restored to that of the parental fae1 line. That no significant increases were observed for transcripts corresponding to key FA synthesis-related genes in badc1,3/fae1/FAH is consistent with the increases being attributed to relief of BADC1 and BADC3-dependent inhibition of ACCase. Thus, data presented here employing badc1,3 null mutants demonstrates both the mechanism of HFA-dependent inhibition of ACCase and an approach to largely mitigating its effects by reducing or eliminating BADC isoforms 1 and 3. That the increased seed oil content in badc1,3/fae1/FAH didn't fully rescue seed weight relative to the parental fae1 line is consistent with previous reports in which the badc1,3 double mutant exhibited a small decrease in seed weight compare to that of wild type seeds, that likely resulted from a buildup of non-esterified FA under conditions in which their supply exceeds cellular demand. Support for this view comes from studies showing excess FAs can be associated with negative cellular consequences, including reductions in axillary bud growth in tobacco (Tso, 1964), microalgal growth (Bosma et al., 2008), cell elongation in Arabidopsis (Li et al., 2011) and cell death in Arabidopsis (Fan et al., 2013; Yang et al., 2015).

The work presented here is an extension of previous studies that focused on understanding mechanisms underlying lipid homeostasis under conditions in which FA supply exceeds that of cellular demand. Using a Brassica napus suspension cell culture we fed FA in the form of Tween esters and monitored reductions in the rate of FAS. Exposure of oleoyl-Tween for up to 2 days resulted in oleoyl-ACP-dependent reversible inhibition of ACCase (Andre et al., 2012); whereas prolonged exposure resulted in irreversible BADC-dependent inhibition (Keereetaweep et al., 2018). That BADC-dependent inhibition of ACCase activity can be elicited by chronic exposure to excesses oleate, a common naturally occurring monounsaturated FA, and ricinoleic acid, a non-native fatty acid, is intriguing. Evidence is accumulating that BADCs are conditional inhibitors of ACCase activity, i.e., that upon the accumulation of excess FA, biotin-lacking, and therefore inactive BADC subunits, replace active BCCP subunits in the BC/BCCP ACCase subcomplex (Salie et al., 2016) (Keereetaweep et al., 2018)(Liu et al., 2019). Based on in vitro studies in which a one-unit pH change caused small changes in the dissociation constants of BADCs and BCCP for BC, it has been proposed that this might contribute to in vivo changes in the inhibition of ACCase related to light- and dark-dependent pH changes (Ye et al., 2020). However, in vivo evidence to support this hypothesis is lacking, and the experiments were conducted under non-physiological conditions. Thus, whether excess FA causes BCCP to dissociate from BC, allowing BADC to join the complex, or whether excess FA drives BADCs into the complex displacing BCCP subunits is an open question that requires additional investigation to resolve.

Due to the desirability of creating an HFA-accumulating variant of a high-yielding crop, work to date has mostly focused on increasing the accumulation of HFA without deleterious effects on seed oil content. Previous studies have shown that negative HFA-dependent deficits including decreased seed oil and seed weight could be mitigated by the overexpression of several common fatty acid accumulation factors. For example, overexpression of OLEOSIN1, a lipid droplet protection protein involved in TAG biosynthesis with FAH was shown to enhance HFA accumulation (Lu et al., 2006). Likewise, overexpression of SEIPIN, a lipid droplet development factor that was previously reported to increase total seed oil (Cai et al., 2015), when expressed in HFA-accumulating seed, increased both total oil and HFA content by more than 60%, likely by increasing LD size and creating a larger sink for TAG-accumulation (Lunn et al., 2018). Seed-specific expression of the WRINKLED1 transcription factor in fae1/FAH restored FA content (Adhikari et al., 2016). Other efforts have focused on the use of factors isolated from species that naturally accumulate modified fatty acid (mFA), in which FA-metabolizing enzymes have evolved preference for mFA. These studies were initially focused on enhancing the transfer of mFA from PC into TAG (Burgal et al., 2008; Kim et al., 2011; van Erp et al., 2011; Hu et al., 2012; Li et al., 2012). In another interesting example, the 18C ricinoleic acid is elongated to the corresponding 20 C lesquerolic acid by a specialized Physaria elongase (Snapp et al., 2014). That lesquerolic acid alleviates feedback inhibition of FAS likely reflects decreased discrimination against lesquerolic relative to ricinoleic in its transfer from PC to TAG. Co-expression of multiple mFA-preferring enzymes, e.g., three castor acyltransferases: GPAT9, LPAT2, and PDAT1A in fae1/FAH seeds resulted in the production of abundant tri-HFA TAG and restored seed oil content relative to the parental fae1 line (Lunn et al., 2019).

The reduced levels of seed oil accumulation reported for HFA-accumulating seed is a general phenomenon common to other mFAs including epoxy (Li et al., 2012), conjugated (Cahoon et al., 2006) and cyclopropane (Yu et al., 2014) FA. The findings presented here demonstrating that knocking out BADC1 and BADC3 in FAH-producing Arabidopsis seeds restored the FA synthesis rate, total FA, seed yield may not be specific for HFA. Indeed, data herein suggests that reducing or eliminating BADC1 and BADC3 gene expression in other mFA-accumulating plants may have similar beneficial effects on mFA accumulation. Further, combining our BADC reduction strategy with the coexpression of other genes, or combinations of genes and/or factors described above will likely increase mFA accumulation to levels equivalent to, or exceeding those of, their natural hosts.

Germination rates typically decline with increasing accumulation levels of mFA (modified fatty acid) accumulation in non-native hosts, even in plants that accumulate normal levels of TAG such as described herein and in previous studies (Lunn et al., 2019). This suggests that mFA generally impair the mobilization of lipid reserves needed for energy production during the critical stages of germination (Lunn et al., 2019). Thus, cellular components that participate in the mobilization mFA-containing TAG, mFA transport and β-oxidation represent additional targets for characterization and expression in non-native hosts to improve cellular energy supplies needed for germination to create robust mFA crops of the future.

We tested the hypothesis that HFA-dependent reduction in FA synthesis can be mediated by BADCs by the introgression of badc1,3 into fae1/FAH. Consistent with the hypothesis, knocking out BADC1 and BADC3 expression increased FA synthesis rates in developing seeds by two-fold, restoring the FA synthesis rate to that of the parental fae1 line. This equally increased both normal FA and HFA accumulation in seeds. The total FA per seed and total oil content in seeds and seeds yield per plant all increased, to an average of 5.8 μg, 37% and 162 mg respectively, compared to 4.9 μg, 33% and 126 mg of fae1/FAH respectively. That fatty acid synthesis-related genes including ACCase subunits, FA condensing enzymes and transcription factors were not significantly increased upon knockout of BADC1 and BADC3, is consistent with the role of BADCs as inhibitors of FA synthesis. Knocking out BADC1 and BADC3 alleviated the inhibition of ACCase, providing a corresponding increase in the FA synthesis rate and steady or improvement in seedling establishment. Combining the deceased expression of BADCs described herein along with the expression of other demonstrated mFA accumulating factors will likely realize the goal of creating crops with industrially relevant levels of HFA-accumulation. This strategy will likely be generalizable to increasing accumulation of many other mFA in seed oils.

The badc1,3/fae1/FAH Arabidopsis showed better establishment than fae1/FAH although their establishment rates are similar. Roots of ten-day old plants were longer and better developed as were aerial parts of the plants. The seed yield per plant was also rescued.

Data herein shows (Example 10) that disruption of badc3 alone in CL37 (fae1/FAH) Arabidopsis increased HFA percentage. Surprisingly, although one expects this disruption to decrease FAS and seed weight and impair seed germination, nonetheless it was empirically determined that the seed weight and seed yield per plant were both increased significantly, and seed germination rate was restored to wild type levels. BADC3 are edited/silenced in specialized fatty acid (sFA) producing crops such as Camelina, soybean and Brassica napus in the same manner as disclosed herein regarding editing/silencing badc1,3. Disruption/silencing of BADC3 in specialized FA-producing crops should lead to increased sFA, crop yield and recovered seed germination.

### EXPERIMENTAL

The following examples are included to demonstrate preferred embodiments of the invention. It should be appreciated by those of skill in the art that the techniques disclosed in the examples, which follow represent techniques discovered by the inventor to function well in the practice of the invention, and thus can be considered to constitute preferred modes for its practice. However, those of skill in the art should, in light of the present disclosure, appreciate that many changes can be made in the specific embodiments, which are disclosed and still obtain a like or similar result without departing from the spirit and scope of the invention.

### Example 1

Materials and Methods

A. Plant Growth Conditions

Arabidopsis badc1,3 double mutant, CL37 (fae1/FAH) and fae1 mutant were used in this study. Seeds were surface sterilized with 70% (v/v) ethanol, followed by 20% (v/v) bleach with 0.01% (v/v) Triton X-100, and washed three to four times with sterile water. Seeds were stratified for 2 d at 4° C. in the dark and germinated on half-Murashige and Skoog (MS) medium supplemented with 1% (w/v) sucrose at 23° C. with a light/dark cycle of 18 h/6 h, photon flux density at 250 μmol m⁻² s⁻¹ plants were grown in walk-in growth chambers at 22° C. with 16 h photoperiod with photon flux density of 70 μmol m⁻² s⁻¹.

B. Seed Germination and Establishment

Seeds of fae1, fae1/FAH, badc1,3/fae1/FAH and badc1,3 were sterilized with ethanol and bleach as described above. A total of 180 seeds in five replicates from each line were sown in plates with ½ MS media containing 1% sucrose under the conditions described above for 14 d. Germination is scored as seeds that produced a radicle, and seedlings that produced roots and green cotyledons were counted as being able to establish (Adhikari et al., 2016).

C. Arabidopsis Cross and Screening of Homozygous Plants

badc1,3/fae1!FAH were generated by crossing the CL37 (frteI!FAH) with badc1,3 double mutant. Homozygous lines were identified by genotyping using PCR coupled with HinfI digestion of PCR products and GC/MS analysis of FA of individual seeds. The genotyping primers used for BADC 1 and 3 are described previously (Keereetaweep et al., 2018). To genotyping fae1, fae1 gene was amplified from CL37 with primer (gFAE-F0: catgagtttgagtatacacatgtcta (SEQ ID NO: 36) and gF AE-R0: aaagaaatcatgtaaacctaaatagaaacgc (SEQ ID NO: 37) and purified for sequencing. According to the fae1 gene sequence information, primer fae-LP: gtgatcgatgagctagagaagaac (SEQ ID NO: 38) and fae-RP: caaggacta TTTGCCGATGCCTTGACATTGCGT AGAGCGAC (SEQ ID NO: 39) were designed to introduce a HinfI restriction site to the fae1 mutant. PCR fragment were restricted with HinfI and the fae1 mutant resulted in two fragments of 200 bp and 40 bp.

D. RNA Extraction and RT-qPCR

RNA from Arabidopsis seeds was extracted according to Wu et al (Wu et al., 2002). RNA quality and concentration were determined by Nanodrop spectroscopy. cDNA was prepared using SuperScript IV VILO Master Mix with ezDNase enzyme (Invitrogen) following manufacture's manual. So Advanced Universal SYBR Green Supermix (Bio-Rad) was used in the reaction mix. RT-qPCR was carried out on the CFX96 Real-time PCR Detection System (Bio-Rad). Gene-specific primers used in the analysis for BADC1 and BADC3 are the same as previously described (Keereetaweep et al., 2018).

FAH-qFI, AATATAGCCATCGCCGCCACCATT (SEQ ID NO: 40) and FAH-qRI: TGGCAAGCAAAGCGA TCGT AAGGT (SEQ ID NO: 41) were used for F AH. The primers used for reference gene UBQIO qF, ACCATCACTTTGGAGGTGGA (SEQ ID NO: 42) and UBQIO qR, GTCAATGGTGTCGGAGCTTT (SEQ ID NO: 43). Statistical analysis of RT-qPCR data was carried out with REST2009 (Pfaffl et al., 200

E. Fatty Acid Analyses

Fatty acid analyses were carried out as described (Broadwater et al., 2002). Lipids were extracted in methanol/chloroform/formic acid (20:10:1) from seeds and heptadecanoic acid (17:0) was added as an internal standard. Total seed lipids were converted into fatty acid methyl esters (FAMEs) in 5% H₂SO₄ in methanol at 90° C. for 60 minutes and extracted with hexane. FAMEs from single seeds were prepared by incubating the seed with 30 μL 0.2M trimethylsulfonium hydroxide in methanol (Butte et al., 1982). Lipid profiles and acyl group identification were analyzed on a Hewlett Packard 6890 gas chromatograph equipped with a 5973 mass selective detector and Agilent DB-FATWAX UI capillary column (30 m×0.25 μm×0.25 μm). The injector was held at 225° C. and the oven temperature was set at 170° C. for one minute and then increased to 250° C. at 10° C./min, finally hold at 250° C. for 7 minutes. The FA percentage values were presented as a mean of at least three biological replicates.

F. [¹⁴C]Acetate Incorporation Assay

[1-¹⁴C]Acetic acid, sodium salt, was purchased from PerkinElmer. Developing seeds at 11-13 days after flower were collected. Approximately 10 mg fresh developing seeds were labeled by incubating in 0.2 mCi of [¹⁴C]acetate for 60 min at room temperature with constant shaking. Cells were subsequently rinsed three times with water. Total lipids were extracted with 500 μL of methanol:chloroform:formic acid (20:10:1, v/v). The organic phase was then extracted with 370 μL of 1 M KCl and 0.2 M H₃PO4 and suspended in 2 mL of Ultima Gold liquid scintillation cocktail (PerkinElmer). The incorporated radioactivity was measured in cpm with a scintillation counter (Packard BioScience).

### Example 2

Generation of Badc1,3/Fae1/FAH Plants

To test the hypothesis that HFA-induced inhibition of fatty acid synthesis results from BADC-dependent inhibition of ACCase, we crossed the badc1,3 double mutant with CL37, a single-insertion homozygous FAH transgenic line in a homozygous mutant fatty acid elongase1 (fae1) background (Kunst L, 1992), the seeds of which are reported to contain 17% HFA (Lu et al., 2006). The level of 18:1, FAH's substrate, is only 13% of TFA in wild type Columbia, therefore fae1, which contains much higher levels (33%) of 18:1 in its seed oil was used. Seeds resulting from this cross were germinated and genetically screened to identify heterozygous badc1,3/fae1/FAH plants. F2 seeds from the heterozygous badc1,3/fae1/FAH plants were planted to screen for homozygous plants which were used for the following studies. The fae1 mutant (Kunst L, 1992), the badc1 and badc3 T-DNA insertion lines (Bohannon and Kleiman, 1978; Bolle et al., 2013) all in the Arabidopsis thaliana Columbia-0 background.

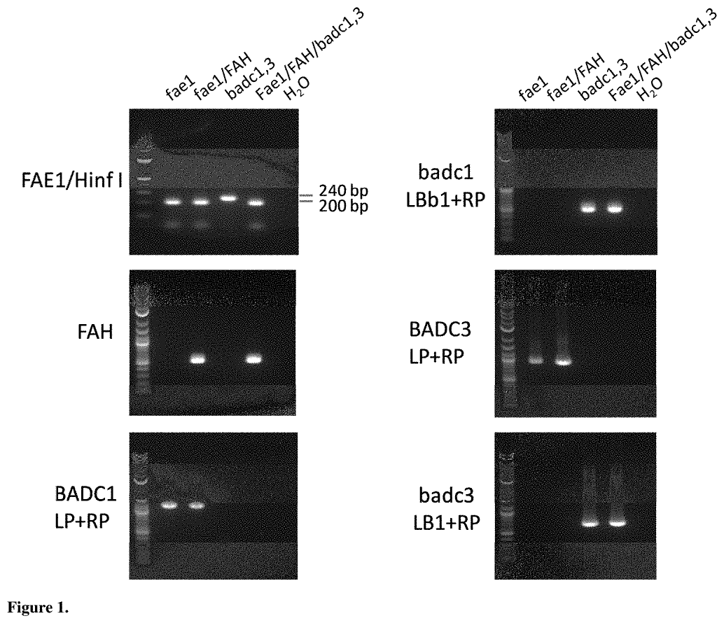

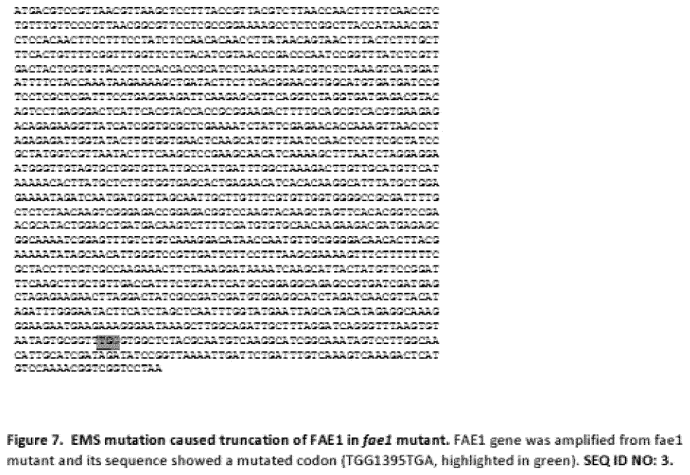

To screen for fae1 homozygous individuals, we first needed to determine the genetic lesion underlying the fae1 mutant. To do this we amplified the fae1 open reading frame from CL37 and sequenced it. We identified a mutation encoding a premature termination at 1395 bp (TGG1393TGA) in the fae1 mutant allele (FIG. 7). We next designed primers to introduce a HinfI restriction site to the PCR amplification of fae1 allele around the mutation site. Subsequent restriction digestion with HinfI of a 240 bp PCR fragment produced two fragments of 200 bp and 40 bp in the fae1 mutant, and only a single 240 bp fragment in the wide type. While the 40 bp fragment is weakly detectable on our gel system, the fae1 mutant displays the 200 bp fragment which can be distinguished from the wild type fragment which is characterized by the larger 240 bp band (FIG. 1).

The genotypes of badc1 or badc3 were determined using gene specific primer pairs in combination with T-DNA specific primer. After screening more than 500 plants 5 badc1,3/fae1 homozygous plants carrying FAH gene were identified. GC/MS analysis of 20 individual seeds for HFA accumulation from each of the 5 badc1,3/fae1 homozygous lines was used to identify FAH expressing homozygotes lines characterized by the accumulation of HFA in all 20 seeds. Finally, we identified two badc1,3/fae1/FAH homozygous individuals.

### Example 3

Knocking Out BADC1 and BADC3 Did not Change FAH Transcription

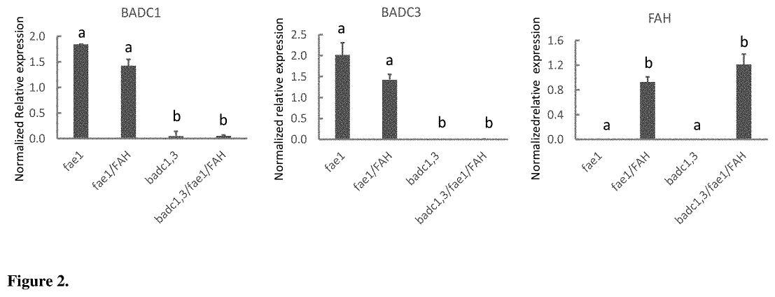

To assess whether badc1,3/fae1/FAH plants were null mutants for BADC1 (AT3G56130) and BADC3 (AT3G15690), we harvested developing seeds from siliques 11 to 13 day after flowering (DAF), and for comparison from fae1, fae1/FAH and badc1,3 seeds grown in parallel. Reverse transcription-quantitative PCR (RT-qPCR) of total RNA extracted from developing seeds confirmed that both BADC1 and BADC3 transcription were dramatically decreased in badc1,3/fae1/FAH and the badc1,3 double mutant (FIGS. 2A and 2B). To evaluate whether knocking out BADC1,3 affects FAH expression, we also quantified FAH transcription. As shown in FIG. 2C, FAH transcription showed no significant change between badc1,3/fae1/FAH and fae1/FAH seeds, showing that knocking out BADC1 and BADC3 genes did not significantly affect FAH expression (FIG. 2C).

### Example 4

Disruption of BADC1 and BADC3 Did not Significantly Alter Transcript Levels of Other FA Synthesis Genes.

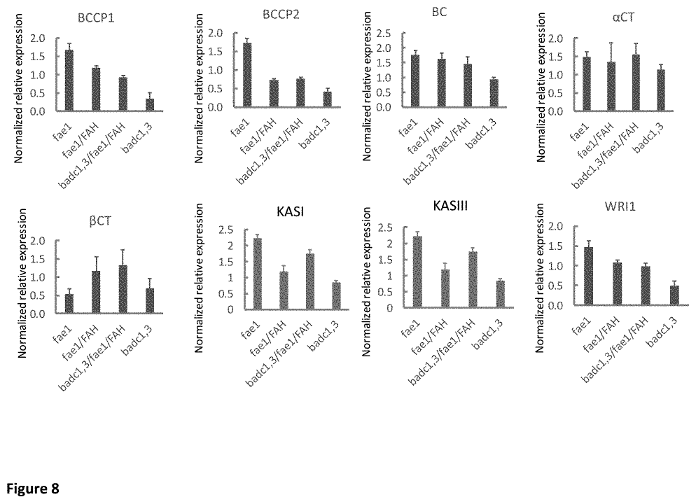

To investigate whether disrupting BADC1 and BADC3 expression affects the transcription of FA synthetic genes, the expression of several genes involved in the FA biosynthetic pathway were quantified by RT-qPCR. Using relative expression (REST)-specific analysis (Pfaffl et al., 2002) designed for comparing qPCR data, no significant changes in transcript abundance were observed for ACCase subunit-encoding genes including BCCP1 (AT5G16390), BCCP2 (AT5G15530), ACCASE BIOTIN CARBOXYLASE (BC, AT5G35360), α-CT (AT2G38040) and β-CT (ATCG00500) or 3-KETOACYL ACP SYNTHASE I (KASI; AT5G46290), and KASIII (AT1G62640) (Maeo et al., 2009; To et al., 2012), two key genes in FA synthesis (FIG. 8). WRI1 was previously shown to regulate a number of FA synthesis genes (Maeo et al., 2009) and all three BADC genes (Liu et al., 2019). Analysis of WRI1 from the same materials showed no significant changes in WRI1 transcript levels (FIG. 8). That the levels of transcripts corresponding to these genes were not significantly different from controls, suggests that the alleviation of inhibition of FA synthesis is not the result of increased transcription of other FA synthesis genes.

### Example 5

Badc1,3/Fae1/FAH Plants Exhibited Increased FA Content and Seed Yield

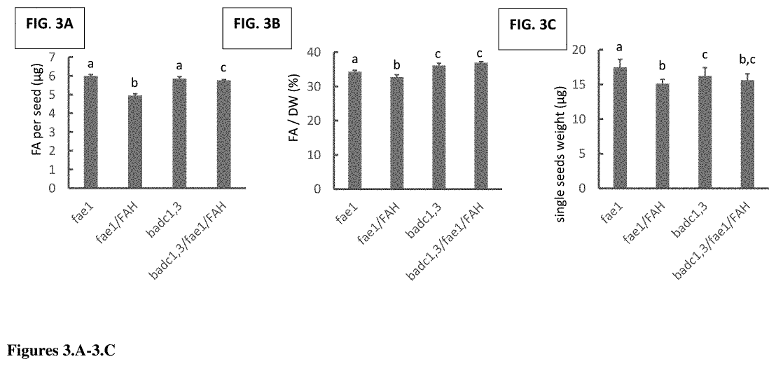

FA content in seeds was quantified to determine if badc1,3 alleviated the feedback inhibition of FA synthesis in seeds with HFA production. fae1 seeds contain 6.00±0.07 μg of total FA, and overexpression of FAH in fae1 significantly reduced FA to 4.94±0.10 g per seed. After introduction of badc1,3, the FA content of the seeds significantly increase by 16.8% to 5.77±0.04 μg per seed (FIG. 3A). Correspondingly, seeds of fae1 plants yielded 34.3±0.4% oil content, expression of FAH significantly decreased the oil content to 32.7±0.7% and the introduction of badc1,3 increased the oil content to 36.9±0.3% (FIG. 3B). The lower oil content in fae1/FAH has been reported to reduce seed weight (Adhikari et al., 2016). Indeed, expression of FAH in fae1 seeds decreased average seed weight from 17.5±1.1 μg to 15.1±0.7 μg (FIG. 3C), but introduction of badc1,3 did not significantly increase seed weight (15.6±1.0 μg per seed). The small significant differences in FA content and seed yield reported herein can be attributed to differences in BADC and FAE gene expression because that both of the T-DNA lines (Bolle et al., 2013) and the fae1 (Kunst L, 1992) line were created in the Arabidopsis Columbia-0 (Arabidopsis Genome, 2000) background.

### Example 6

Both HFA and Unmodified FA Increased in Badc1,3/Fae1/FAH

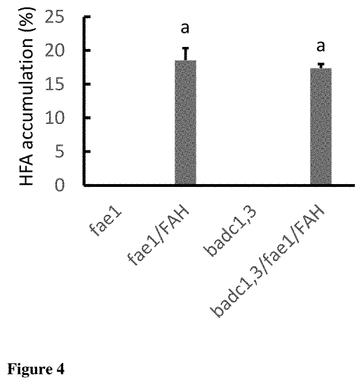

The badc1,3 double mutant increased total FA in badc1,3/fae1/FAH seeds. To determine whether the increase of FA was specific for either unmodified FAs or HFAs, FAMEs from the respective seed backgrounds were analyzed. HFA in fae1/FAH and badc1,3/fae1/FAH were 18.6±1.8% and 17.4 f 0.6% of the total FAs respectively (FIG. 4), showing that badc1,3 didn't significantly change the HFA percentage in mature seeds (student t test, p>0.05), rather, the increases are in both HFAs and native FAs.

### Example 7

FA Synthesis Rate is Restored in Badc1,3/Fae1/FAH

It was previously reported that the production of HFA in fae1 seeds expressing FAH was associated with a reduced rate of de novo FA synthesis that resulted in the observed decrease in oil content compared with the fae1 parental line (Bates et al., 2014). The introduction of badc1,3 in the fae1/FAH line restored the FA content suggesting that it had alleviated the previously observed inhibition of FA synthesis reported in non-HFA producing lines (Salie et al., 2016; Keereetaweep et al., 2018). To test this hypothesis, mid-phase developing seeds 11-13 days after flowering were collected and their fatty acid synthesis rates were determined by measuring the rate of [¹⁴C]acetate incorporation into FAs by total lipid extraction and scintillation counting. We first validated the assay by showing linear incorporation of [¹⁴C]acetate between 20 and 100 minutes using badc1,3 seeds (FIG. 9) and chose a 60 minute incubations for subsequent experiments. As shown in FIG. 5, compared to fae1, the badc1,3 double mutant showed a 36.8% increase in fatty acid synthesis rate, whereas expression of FAH in fae1 decreased fatty acid synthesis rate by 52.2% with respect to that of fae1. When FAH was expressed in badc1,3/fae1, the fatty acid synthesis rate was fully restored to that of parental fae1 seeds.

### Example 8

Seed Germination and Development

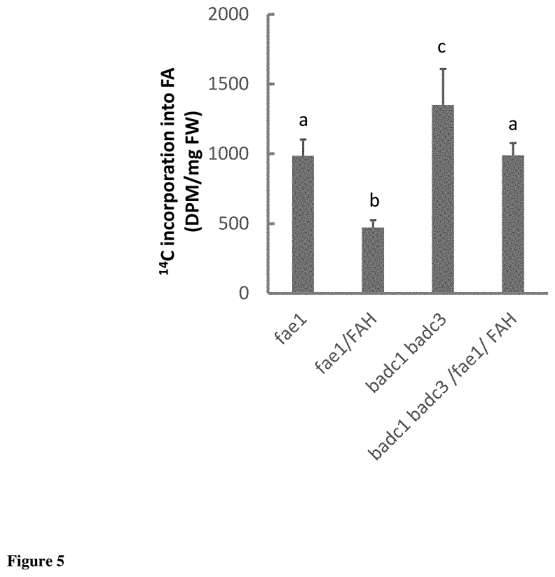

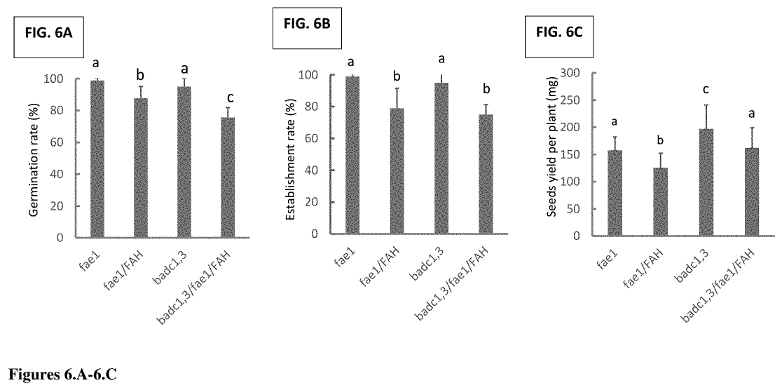

Overexpression of FAH in fae1 has been reported to decrease seed germination (Adhikari et al., 2016; Lunn et al., 2018; Lunn et al., 2018). To test if the restored FA content in badc1,3 can mitigate the germination defects, seeds of badc1,3/fae1/FAH were tested for germination and seedling establishment relative to the fae1/FAH, badc1,3 parental lines and fae1. Emergence of the radicle was used as a germination marker, and the appearance of roots and green cotyledons was used as a marker for establishment. Germination of fae1/FAH lines was reduced to 88% compared with 99% for fae1 (FIG. 6A). The germination rate of badc1,3/fae1/FAH was even lower than fae1/FAH at 76%. badc1,3 showed a germination rate of 95%, i.e., similar to that of fae1. The seedling establishment rates of fae1 and badc1,3 were the same as their germination rates (FIG. 6B). 90% of geminated fae1/FAH seedlings continued to establishment, whereas 99% of germinated badc1,3/fae1/FAH seeds continued to establishment, resulting in similar establishment rates with respect to all seeds for these two genotypes. Comparison of seedling establishment rates at 7 and 10 days showed the combining badc1,3 with fae1/FAH had the effect of reducing germination while increasing seedling establishment (FIG. 10). While the growth rate of badc1,3/fae1/FAH was higher than that of fae1/FAH, at maturity no visible differences were observed with respect to plant height and leaf size. However, fae1 plants produced 158 mg of seeds per plant, which decreased to 126 mg in fae1/FAH, while the introduction of badc1,3 in the fae1/FAH lines more than compensated, increasing seed yield per plant to 162 mg (FIG. 6C). In summary, combining badc1,3 with fae1/FAH improved seedling establishment and restored seed yield.

### Example 9

Increasing Specialty Oil in Exemplary Camelina Crop Plants.

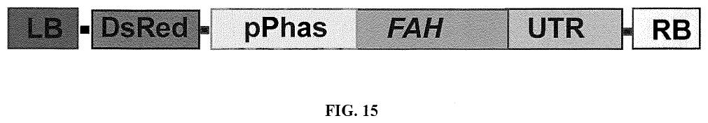

We use a fad2/fae1 Camelina background generated through RNAi suppression of FAD2 and FAE1 that accumulates over 60% 18:1 FA in mature seed (Nguyen et al. (2013)) (see FIGS. 16Q and 16R Arabidopsis sequences). RcFAH gene is placed under the control of seed-specific phaseolin promoter (see vector diagram of FIG. 15) and transformed into fad2/fae1 Camelina. Independently transformed lines are analyzed by gas chromatography-linked mass spectrometry (GC-MS) to determine their lipid composition. Homozygous lines with high HFA accumulation are chosen for disruption of BADC 1 and BADC3 gene expression with the use of CRISPR/Cas9 gene editing. Camelina is a hexaploid so the following target sites are identified to simultaneously disrupt all 3 isoforms of each gene: i.e, either CGGTGGAGATTATCCAACAG (SEQ ID NO: 44) or TTATGGTGATCCTCTGGTTG (SEQ ID NO: 45) are used as the target site for editing Camelina BADC1, including Csa04g042500.1, Csa06g030800.I and Csa09g068300.I; and AAAATTAAAATCTCAGCAGT (SEQ ID NO: 46) is the target site for editing Camelina BADC3 including Csa15g020290.1, Csa19g022480.1 and Csa01g018320.1. The transgenic seeds are screened in media supplemented with Hygromycin B and DNA is extracted and BADC 1 and BADC3 genes are amplified and sequenced to identify the CRISPR/cas9-induced lesion and verify the target gene disruption. The fatty acid synthesis rate in developing seed of fad2/fae1 !FAHIbadc1,3 is evaluated. Mature seeds are collected, seed size and seed weight are measured, and fatty acid methyl esters are prepared from the seeds for GC-MS analysis for FA composition and total FA quantification.

### Example 10

Disruption of Specific BADC Gene(s) Increases Both Hydroxy Fatty Acid Accumulation and Seed Germination in Plants Expressing a Fatty Acid Hydroxylase FAH Enzyme

In order to test whether knock out either BADC or BADC3 effects hydroxy fatty acid (HFA) accumulation in plants, we used a CRISPR/cas9 strategy to disrupt single BADC genes in Arabidopsis Columbia-0 line CL37 comprising a mutation in fatty acid elongase 1 (fae1) and overexpressing the Ricinus communis fatty acid hydroxylase gene (FAH), that accumulates approximately 17% HFA in seeds. Target sites in the exon of BADC1 and 3 were chosen using online genome-wide prediction of plant CRISPR/Cas9 target sites, and target specificities were evaluated on the website of potential off-target finder. Finally, we constructed two vectors, one targeting the 2^{nd} and 5^{th} exons of BADC1, the other targeting the 2^{nd} and 4^{th} exons of BADC3 and transformed each into CL37. Transformed progenies were genotyped and sequence analysis confirmed editing of BADC1 or BADC3 had resulted four CL37 lines with truncated BADC1 (CL37/badc1) and 4 lines with truncated BADC3 (CL37/badc3).

A. CL37/Badc3 Plants Exhibited Increased HFA Content

FA composition in seeds was analyzed to determine if badc1 or badc3 influenced HFA accumulation. CL37 seeds contain 19.1% HFA, 4 CL37/badc1 lines showed similar percentage of HFA, implying disrupting BADC1 did not change HFA accumulation. Surprisingly, disruption of badc3 significantly increased the HFA percentage in all 4 CL37/badc3 lines by more than 30% with values ranging from 26.1 to 27.2% (FIG. 11). Notably, the increased HFA percentage didn't further inhibit FA synthesis, CL37/badc3 retained similar ACCase activity as that of CL37.

B. CL37/Badc3 Rescued Seed Germination and Increased Seed Yield

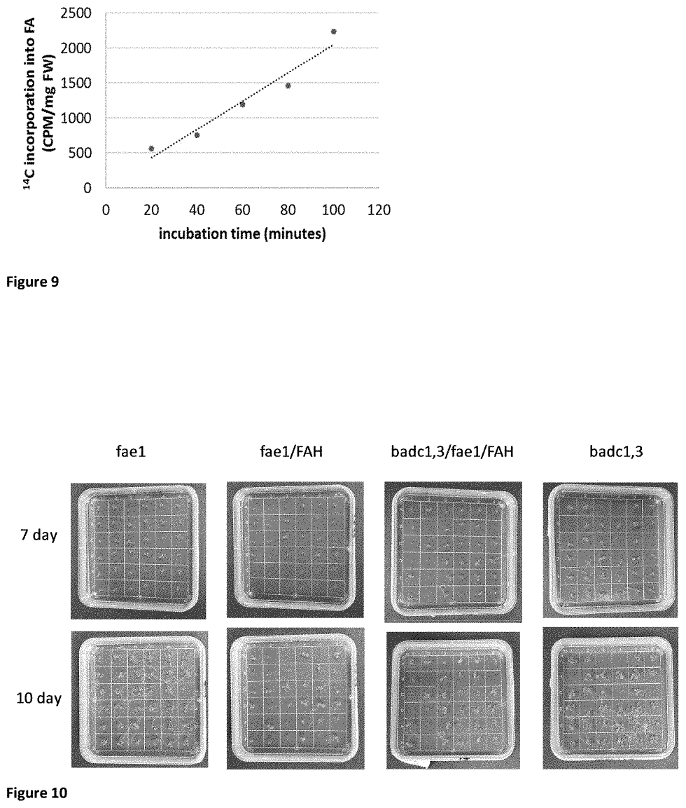

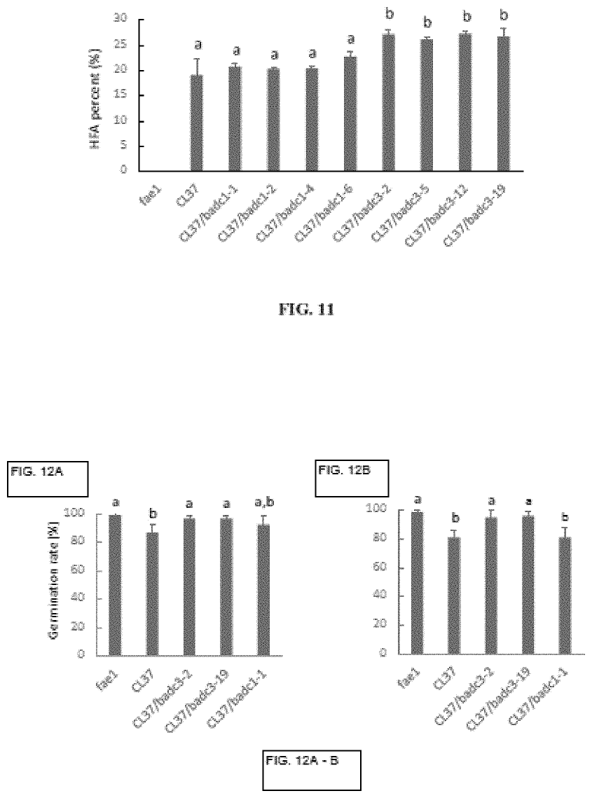

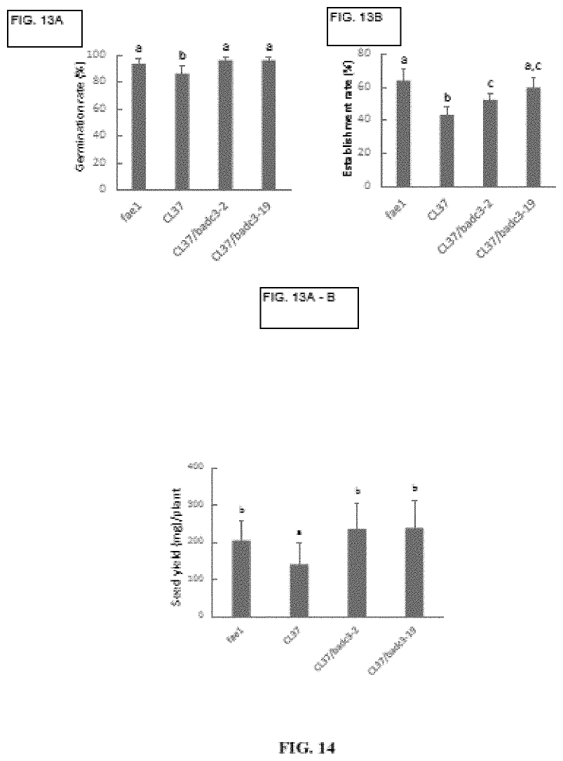

Overexpression of FAH in fae1 is reported to decrease seed germination (Adhikari et al., 2016; Lunn et al., 2018; Lunn et al., 2018). To test if increased HFA in CL37/badc3 seeds would further impair seed germination, seeds of CL37/badc3 were tested for germination and seedling establishment relative to CL37 and fae1. Emergence of the radicle was used as a germination marker, and the appearance of roots and green cotyledons was used as a marker for establishment. On plates comprising ½ MS supplemented with sucrose plate, germination of CL37 lines was 87% compared with 99% for fae1 (FIG. 12A). Surprisingly, the germination rate of CL37/badc3 was much higher than CL37 at 97%. The seedling establishment rates of fae1 and CL37/badc3 were similar to their germination rates (FIG. 12B), whereas establishment rates dropped to 81% in both CL37 and CL37/badc1. Consistently, seed germination and seedling establishment in % MS plate without sugar were also rescued in CL37/badc3 line (FIG. 13A-B). While the growth rate of CL37/badc3 was similar as that of CL37, no visible differences were observed with respect to plant height and leaf size during plants growth and maturity. However, fae1 plants produced 205 mg of seeds per plant, which decreased to 141 mg in CL37, while disruption of badc3 in the CL37 lines increased seed yield per plant by 67%, to more than 235 mg, that is an increase of 15% relative to fae1 (FIG. 14). In summary, disruption of badc3 in CL37 rescued seed germination and seedling establishment, and significantly increased seed yield.

### REFERENCES

Adhikari N D, Bates P D, Browse J (2016) WRINKLED1 Rescues Feedback Inhibition of Fatty Acid Synthesis in Hydroxylase-Expressing Seeds. Plant Physiol 171: 179-191
Andre C, Haslam R P, Shanklin J (2012) Feedback regulation of plastidic acetyl-CoA carboxylase by 18:1-acyl carrier protein in Brassica napus. Proc Natl Acad Sci USA 109: 10107-10112
Arabidopsis Genome I (2000) Analysis of the genome sequence of the flowering plant Arabidopsis thaliana. Nature 408: 796-815
Bates P D, Johnson S R, Cao X, Li J, Nam J W, Jaworski J G, Ohlrogge J B, Browse J (2014) Fatty acid synthesis is inhibited by inefficient utilization of unusual fatty acids for glycerolipid assembly. Proc Natl Acad Sci USA 111: 1204-1209
Bohannon M B, Kleiman R (1978) Cyclopropene Fatty-Acids of Selected Seed Oils from Bombacaceae, Malvaceae, and Sterculiaceae. Lipids 13: 270-273
Bolle C, Huep G, Kleinbolting N, Haberer G, Mayer K, Leister D, Weisshaar B (2013) GABI-DUPLO: a collection of double mutants to overcome genetic redundancy in Arabidopsis thaliana. Plant J 75: 157-171
Bosma R, Miazek K, Willemsen S M, Vermue M H, Wijffels R H (2008) Growth inhibition of Monodus subterraneus by free fatty acids. Biotechnol Bioeng 101: 1108-1114
Broadwater J A, Whittle E, Shanklin J (2002) Desaturation and hydroxylation. Residues 148 and 324 of Arabidopsis FAD2, in addition to substrate chain length, exert a major influence in partitioning of catalytic specificity. J Biol Chem 277: 15613-15620
Burgal J, Shockey J, Lu C, Dyer J, Larson T, Graham I, Browse J (2008) Metabolic engineering of hydroxy fatty acid production in plants: RcDGAT2 drives dramatic increases in ricinoleate levels in seed oil. Plant Biotechnol J 6: 819-831
Butte W, Eilers J, Hirsch K (1982) Trialkysulfonium-hydroxides and trialkylselonium-hydroxides for the pyrolytic alkylation of acidic compounds. Anal. Lett. 15: 841-850
Cahoon E B, Dietrich C R, Meyer K, Damude H G, Dyer J M, Kinney A J (2006) Conjugated fatty acids accumulate to high levels in phospholipids of metabolically engineered soybean and Arabidopsis seeds. Phytochemistry 67: 1166-1176
Cahoon E B, Shockey J M, Dietrich C R, Gidda S K, Mullen R T, Dyer J M (2007) Engineering oilseeds for sustainable production of industrial and nutritional feedstocks: solving bottlenecks in fatty acid flux. Current Opinion in Plant Biology 10: 236-244
Cai Y, Goodman J M, Pyc M, Mullen R T, Dyer J M, Chapman K D (2015) Arabidopsis SEIPIN Proteins Modulate Triacylglycerol Accumulation and Influence Lipid Droplet Proliferation. Plant Cell 27: 2616-2636
Fan J, Yan C, Xu C (2013) Phospholipid:diacylglycerol acyltransferase-mediated triacylglycerol biosynthesis is crucial for protection against fatty acid-induced cell death in growing tissues of Arabidopsis. Plant J 76: 930-942
Feria Bourrellier A B, Valot B, Guillot A, Ambard-Bretteville F, Vidal J, Hodges M (2010) Chloroplast acetyl-CoA carboxylase activity is 2-oxoglutarate-regulated by interaction of PII with the biotin carboxyl carrier subunit. Proc Natl Acad Sci USA 107: 502-507
Haslam R P, Ruiz-Lopez N, Eastmond P, Moloney M, Sayanova O, Napier J A (2013) The modification of plant oil composition via metabolic engineering-better nutrition by design. Plant Biotechnology Journal 11: 157-168
Hu Z H, Ren Z H, Lu C F (2012) The Phosphatidylcholine Diacylglycerol Cholinephosphotransferase Is Required for Efficient Hydroxy Fatty Acid Accumulation in Transgenic Arabidopsis. Plant Physiology 158: 1944-1954
Keereetaweep J, Liu H, Zhai Z, Shanklin J (2018) Biotin Attachment Domain-Containing Proteins Irreversibly Inhibit Acetyl CoA Carboxylase. Plant Physiol 177: 208-215
Kim H, Gardner H W, Hou C T (2000) Production of isomeric 9,10,13 (9,12,13)-trihydroxy-11E (10E)-octadecenoic acid from linoleic acid by Pseudomonas aeruginosa PR3. Journal of Industrial Microbiology & Biotechnology 25: 109-115
Kim H U, Lee K R, Go Y S, Jung J H, Suh M C, Kim J B (2011) Endoplasmic Reticulum-Located PDAT1-2 from Castor Bean Enhances Hydroxy Fatty Acid Accumulation in Transgenic Plants. Plant and Cell Physiology 52: 983-993
Kunst L T D, Underhill E W (1992) Fatty acid elongation in developing seeds of Arabidopsis thaliana Plant Physiol Biochem 30: 425-434
Li M, Bahn S C, Guo L, Musgrave W, Berg H, Welti R, Wang X (2011) Patatin-related phospholipase pPLAIIIbeta-induced changes in lipid metabolism alter cellulose content and cell elongation in Arabidopsis. Plant Cell 23: 1107-1123
Li R, Yu K, Wu Y, Tateno M, Hatanaka T, Hildebrand D F (2012) Vernonia DGATs can complement the disrupted oil and protein metabolism in epoxygenase-expressing soybean seeds. Metab Eng 14: 29-38
Liu H, Zhai Z, Kuczynski K, Keereetaweep J, Schwender J, Shanklin J (2019) WRINKLED1 Regulates BIOTIN ATTACHMENT DOMAIN-CONTAINING Proteins that Inhibit Fatty Acid Synthesis. Plant Physiol 181: 55-62
Lu C, Fulda M, Wallis J G, Browse J (2006) A high-throughput screen for genes from castor that boost hydroxy fatty acid accumulation in seed oils of transgenic Arabidopsis. Plant J 45: 847-856
Lu C F, Xin Z G, Ren Z H, Miquel M, Browse J (2009) An enzyme regulating triacylglycerol composition is encoded by the ROD1 gene of Arabidopsis. Proceedings of the National Academy of Sciences of the United States of America 106: 18837-18842
Lunn D, Smith G A, Wallis J G, Browse J (2018) Development Defects of Hydroxy-Fatty Acid-Accumulating Seeds Are Reduced by Castor Acyltransferases. Plant Physiol 177: 553-564
Lunn D, Wallis J G, Browse J (2018) Overexpression of Seipin1 Increases Oil in Hydroxy Fatty Acid-Accumulating Seeds. Plant Cell Physiol 59: 205-214
Lunn D, Wallis J G, Browse J (2019) Tri-Hydroxy-Triacylglycerol Is Efficiently Produced by Position-Specific Castor Acyltransferases. Plant Physiol 179: 1050-1063
Maeo K, Tokuda T, Ayame A, Mitsui N, Kawai T, Tsukagoshi H, Ishiguro S, Nakamura K (2009) An AP2-type transcription factor, WRINKLED1, of Arabidopsis thaliana binds to the AW-box sequence conserved among proximal upstream regions of genes involved in fatty acid synthesis. Plant J 60: 476-487
Napier J A (2007) The production of unusual fatty acids in transgenic plants. Annu Rev Plant Biol 58: 295-319
Nguyen et al. (2013) Plant Biotechnol J 11: 759-769)
Pfaffl M W, Horgan G W, Dempfle L (2002) Relative expression software tool (REST) for group-wise comparison and statistical analysis of relative expression results in real-time PCR. Nucleic Acids Res 30: e36
Salie M J, Zhang N, Lancikova V, Xu D, Thelen J J (2016) A Family of Negative Regulators Targets the Committed Step of de Novo Fatty Acid Biosynthesis. Plant Cell 28: 2312-2325
Shivaiah K K, Ding G, Upton B, Nikolau B J (2020) Non-Catalytic Subunits Facilitate Quaternary Organization of Plastidic Acetyl-CoA Carboxylase. Plant Physiology 182: 756-775
Snapp A R, Kang J, Qi X, Lu C (2014) A fatty acid condensing enzyme from Physaria fendleri increases hydroxy fatty acid accumulation in transgenic oilseeds of Camelina sativa. Planta 240: 599-610
To A, Joubes J, Barthole G, Lecureuil A, Scagnelli A, Jasinski S, Lepiniec L, Baud S (2012) WRINKLED transcription factors orchestrate tissue-specific regulation of fatty acid biosynthesis in Arabidopsis. Plant Cell 24: 5007-5023
Tso T C (1964) Plant-Growth Inhibition by Some Fatty Acids and Their Analogues. Nature 202: 511-512
van de Loo F J, Broun P, Turner S, Somerville C (1995) An oleate 12-hydroxylase from Ricinus communis L. is a fatty acyl desaturase homolog. Proc. Natl. Acad. Sci. USA 92: 6743-6747
van Erp H, Bates P D, Burgal J, Shockey J, Browse J (2011) Castor phospholipid:diacylglycerol acyltransferase facilitates efficient metabolism of hydroxy fatty acids in transgenic Arabidopsis. Plant Physiol 155: 683-693
Vanhercke T, Wood C C, Stymne S, Singh S P, Green A G (2013) Metabolic engineering of plant oils and waxes for use as industrial feedstocks. Plant Biotechnology Journal 11: 197-210
Wang X, Li L, Zheng Y, Zou H, Cao Y, Liu H, Liu W, Xian M (2012) Biosynthesis of long chain hydroxyfatty acids from glucose by engineered Escherichia coli. Bioresour Technol 114: 561-566
Wu Y R, Llewellyn D J, Dennis E S (2002) A quick and easy method for isolating good-quality RNA from cotton (Gossypium hirsutum L.) tissues. Plant Molecular Biology Reporter 20: 213-218
Yang Y, Munz J, Cass C, Zienkiewicz A, Kong Q, Ma W, Sanjaya, Sedbrook J, Benning C (2015) Ectopic Expression of WRINKLED1 Affects Fatty Acid Homeostasis in Brachypodium distachyon Vegetative Tissues. Plant Physiol 169: 1836-1847
Ye Y, Fulcher Y G, Sliman D J, 2nd, Day M T, Schroeder M J, Koppisetti R K, Bates P D, Thelen J J, Van Doren S R (2020) The BADC and BCCP subunits of chloroplast acetyl-CoA carboxylase sense the pH changes of the light-dark cycle. J Biol Chem
Yu X H, Prakash R R, Sweet M, Shanklin J (2014) Coexpressing Escherichia coli cyclopropane synthase with Sterculia foetida Lysophosphatidic acid acyltransferase enhances cyclopropane fatty acid accumulation. Plant Physiol 164: 455-465

Each and every publication and patent mentioned in the above specification is herein incorporated by reference in its entirety for all purposes. Various modifications and variations of the described methods and system of the invention will be apparent to those skilled in the art without departing from the scope and spirit of the invention. Although the invention has been described in connection with specific embodiments, the invention as claimed should not be unduly limited to such specific embodiments. Indeed, various modifications of the described modes for carrying out the invention which are obvious to those skilled in the art and in fields related thereto are intended to be within the scope of the following claims.

1. A transgenic plant or part thereof that comprises:
a) a genomic mutation selected from the group consisting of a mutation of fad2, fad3, and fae1, or any combination of such mutations,
b) the reduced expression of one or both endogenous BADC1 and BADC3 genes, and
c) one or more transgenes that alter metabolism of a target fatty acid.

2. The transgenic plant or part thereof of claim 1, wherein said transgenic plant part comprises wild type BADC1 gene and reduced expression of said endogenous BADC3 gene.

3. The transgenic plant or part thereof of claim 1, wherein said transgenic plant part comprises reduced expression of said endogenous BADC1 gene and BADC3 gene.

4. The transgenic plant or part thereof of claim 1, wherein said plant is Camelina sativa, Brassica napus or Glycine max.

5. The transgenic plant or part thereof of claim 1, wherein said genomic mutation is fad2/fae1 or fad3/fae1.

6. The transgenic plant or part thereof of claim 1, wherein said one or more transgenes encode Ricinus fatty acid hydroxylase (FAI H), E. coli cyclopropane fatty acid synthase, Crepis palaestina delta 12 fatty acid epoxygenase, Crepis alpina delta-12 fatty acid acetylenase, or Momordica charantia Conjugase (FadX).

7. The transgenic plant or part thereof of claim 1, wherein said one or more transgenes are under control of a seed-specific promoter.

8. The transgenic plant or part thereof of claim 1, wherein said target fatty acid comprises one or more of hydroxyl fatty acids, medium-chain fatty acids, very-long-chain fatty acids (VLCFAs), monounsaturated fatty acids (MUFAs), γ-linolenic acid, stearidonic acids, α-eleostearic acid, conjugated fatty acids, epoxy fatty acids, cyclic fatty acids and acetylenic fatty acids.

9. The transgenic plant or part thereof of claim 1, wherein said transgenic plant part is from Camelina sativa, Brassica napus or Glycine max, said genomic mutation is fad3 fae1, and said transgene encodes acetylanase, conjugase, epoxygenase or any combinations thereof.

10. The transgenic plant or part thereof of claim 1, wherein said transgenic plant part is from Camelina sativa, Brassica napus or Glycine max, said genomic mutation is fad2/fae1, and said transgene encodes Ricinus fatty acid hydroxylase.

11. The transgenic plant or part thereof of claim 1, wherein said transgenic plant part is from Camelina sativa, Brassica napus or Glycine max, said genomic mutation is fae1, and said transgene encodes Ricinus fatty acid hydroxylase.

12. The transgenic plant or part of claim 1, wherein said reduced expression comprises complete silencing.

13. The transgenic plant or part of claim 2, wherein said reduced expression comprises complete silencing.

14. The transgenic plant or part of claim 3, wherein said reduced expression comprises complete silencing.

15. A progeny plant of the transgenic plant of claim 1.

16. A transgenic seed that produces the transgenic plant of claim 1.

17. A method of producing the target fatty acid using the transgenic plant part of claim 1.


SEQUENCE LISTING (46 sequences)

SEQ ID NO: 1 - protein, 281 aa, Arabidopsis thaliana
MASSAALGSLHQTLGSAINSQSEVHSLSGNWSASGNSCVPRWRLSNRNSNYRLVLRAKAAKSSTTTISDGSSDASVSDGKKTVRRITFPKEVEALVHEMCDETEVAVLQLKVGDFEMNLKRKIGAATNPIPVADISPTVAPPIPSEPMNKSASSAPSPSQAKPSSEKVSPFKNTSYGKPAKLAALEASGSTNYVLVTSPAVGKFQRSRTVKGKKQSPSCKEGDAIKEGQVIGYLHQLGTELPVTSDVAGEVLKLLSDDGDSVGYGDPLVAVLPSFHDINIQ

SEQ ID NO: 2 - DNA, 2613 bp, Arabidopsis thaliana
acggaccgtagtgtagtagtagatgcggcggacggagttaccaaagaagaaggccgctcaaaataattaaatttgttcaaccgtcatcttcttcaactgatcttagctcaactaacacactctttcttcttggcgtcaattcaatcaaccaaaacctttttctcctatctagctcacgctttcttcttcttccaatggcgtcttctgcagctctcggatctctccatcgtgagtctcttgctctctcactctctgcgttttacttattctgttgatttcatgaggataggaaaactagaaatggaggaccatgagtaaaatttcggaaatgaaaggcatagattggagctatccgttagtgacgttgttgcttcttagagtgtaatttagcgacttaattaagtttcaatctcggatcttgtgtgtctaatttgtatcaagagatgtttcagctagaaaaagtgaattttatttgttccattttacagagactttagggtcagccattaattcacagagtgaggttcactcgctttctggaaactggtctgcctctggtaattcatgtgtgccacggtggagattatccaacaggaacagcaactacaggctcgtgttacgtgctaaggccgctaaatcttcgacaacaaccataagtgatggtgagtttatcttccacaattcttcttcatgttcattttgctggttaatgcctttttttactatcaggcttgtctcatcactttgctaatacgatccattggccaaagatggtattaatctgttttgctcttaaaggaactggaactgaaattcttagtctcgtttgctcttaaaggaactagaaatgtaactgtaagcctcggtcttttaccagctgtcttggaaccaggagatacagtgatgtgatattggaacatttttcttttatttcctatgacttttgcttatttttggctttgcaggttcatctgatgctagtgtgtcagacgggaagaaaacagttcgacggataactttcccgaaagaagtggaggtttcttccttgcctttcatgggtcttagatattaggttctttaatttataagtttggtaggtgatgatatgacgatttcctcaaatatgcactttctagatcgtcaggattttggatgcataacttcaggcaatctactcttataatttttaatcatgacgtatggatgtacctttctttatatgttgttagatgaaatgttgcaggcactggttcacgagatgtgtgatgaaactgaggttgctgtgctgcaacttaaggcaagtctctctgccattagttttaacttcattattattattatttgtaaactttctttgaggctactacaaagacgagtgcattttactcaaccaacaatatggggctaaatatcactgatttgagataatagaatgtaggttccaacaattagactcttctgaagctttcttttgctacaggttggagatttcgagatgaacctaaaacggaagattggagcagccacaaaccccattcccgtggcggatatatctccaactgtagcgcctcctattccttctgaacctatgaataaatctgcttcttcggctcctagcccatctcaagcaaagccttcctctgagaaagtgtctccatttaagaatacatcatatgggaaaccagcaaagttggctgctttggaggcatctggatccaccaactatgtgttagtcacatctcccgcagtatgagatccatttcctaattagtggttgctttcatatcccttaatttctctgcagttttcttgtttgatttgatcttgtttcttctcttaccaaaaggtgggcaagtttcagaggagcagaactgtaaaaggaaagaaacaatctcctagctgcaaagaggtaaacgactctaaattcttttgcatctcttagaacaaaagaacagaaataagatcaaagagctaagtgaaaaaaactccttagggtgatgcaataaaggaaggccaagttattggatacttacatcagttgggaacagaacttccagtgacggtaatatcttaactaatatatccatctcttctttgaaactatctaatcagactcatcgatcttgctatttgtcgagcagtcagatgtagctggagaagtccttaagcttctttcagatgacggaggtaaacatttgagtattcaaaccgtttcatttagtatgaacattcagaaattatataagtgaattgatatgaactcatgcttcgtgtgcaaacagactccgtaggttatggagatcctctggttgcagtcttgccatctttccacgacatcaacatccagtgatgatggtttcttcagcccaattccatagcaaatgaatagtctttcatccggagactgtactattcatcttctcctgtgtttgttcaatgaagatttgtaatctgtttagttgcaaagagtctactttgatcttgctctcatcatttgtcacgtaatgtggattttctgcaccagagaaaaaaaacaattgtggaatttttatagaaatgacgtggctatcttatcttctccgatcatcaaataaaatcaaggctcaaaaattc

SEQ ID NO: 3 - DNA, 1521 bp, Artificial Sequence
  Synthetic
atgacgtccgttaacgttaagctcctttaccgttacgtcttaaccaactttttcaacctctgtttgttcccgttaacggcgttcctcgccggaaaagcctctcggcttaccataaacgatctccacaacttcctttcctatctccaacacaaccttataacagtaactttactctttgctttcactgttttcggtttggttctctacatcgtaacccgacccaatccggtttatctcgttgactactcgtgttaccttccaccaccgcatctcaaagttagtgtctctaaagtcatggatattttctaccaaataagaaaagctgatacttcttcacggaacgtggcatgtgatgatccgtcctcgctcgatttcctgaggaagattcaagagcgttcaggtctaggtgatgagacgtacagtcctgagggactcattcacgtaccaccgcggaagacttttgcagcgtcacgtgaagagacagagaaggttatcatcggtgcgctcgaaaatctattcgagaacaccaaagttaaccctagagagattggtatacttgtggtgaactcaagcatgtttaatccaactccttcgctatccgctatggtcgttaatactttcaagctccgaagcaacatcaaaagctttaatctaggaggaatgggttgtagtgctggtgttattgccattgatttggctaaagacttgttgcatgttcataaaaacacttatgctcttgtggtgagcactgagaacatcacacaaggcatttatgctggagaaaatagatcaatgatggttagcaattgcttgtttcgtgttggtggggccgcgattttgctctctaacaagtcgggagaccggagacggtccaagtacaagctagttcacacggtccgaacgcatactggagctgatgacaagtcttttcgatgtgtgcaacaagaagacgatgagagcggcaaaatcggagtttgtctgtcaaaggacataaccaatgttgcggggacaacacttacgaaaaatatagcaacattgggtccgttgattcttcctttaagcgaaaagtttctttttttcgctaccttcgtcgccaagaaacttctaaaggataaaatcaagcattactatgttccggatttcaagcttgctgttgaccatttctgtattcatgccggaggcagagccgtgatcgatgagctagagaagaacttaggactatcgccgatcgatgtggaggcatctagatcaacgttacatagatttgggaatacttcatctagctcaatttggtatgaattagcatacatagaggcaaagggaagaatgaagaaagggaataaagcttggcagattgctttaggatcagggtttaagtgtaatagtgcggtttgggtggctctacgcaatgtcaaggcatcggcaaatagtccttggcaacattgcatcgatagatatccggttaaaattgattctgatttgtcaaagtcaaagactcatgtccaaaacggtcggtcctaa

SEQ ID NO: 4 - DNA, 1164 bp, Ricinus communis
atgggaggtggtggtcgcatgtccactgtcataaccagcaacaacagtgagaagaaaggaggaagcagccaccttaagcgagcgccgcacacgaagcctcctttcacacttggtgacctcaagagagccatcccaccccattgctctgaacgctcttttgtgcgctcattctcctatgttgcctatgatgtctgcttaagttttcttttctactcgatcgccaccaacttcttcccttacatctcttctccgctctcgtatgtcgcttggctggtttactggctcttccaaggctgcattctcactggtctttgggtcatcggccatgaatgtggccatcatgcttttagtgagtatcagctggctgatgacattgttggcctaattgtccattctgcacttctggttccatatttttcatggaaatatagccatcgccgccaccattctaacataggatctctcgagcgagacgaagtgttcgtcccgaaatcaaagtcgaaaatttcatggtattctaagtacttaaacaacccgccaggtcgagttttgacacttgctgccacgctcctccttggctggcctttatacttagctttcaatgtctctggtagaccttacgatcgctttgcttgccattatgatccctatggcccaatattttccgaaagagaaaggcttcagatttacattgctgacctcggaatctttgccacaacgtttgcgctttatcaggctacaatggcaaaagggttggcttgggtaatgcgtatctatggggtgccattgcttattgttaactgtttccttgttatgatcacatacttgcagcacactcacccagctattccacgctatggctcatcggaatgggattggctccggggagcaatggtgactgtcgatagagattatggggtgttgaataaagtattccataacattgcagacactcaggtagctcatcatctctttgctacagtgccacattaccatgcaatggaggccactaaagcaatcaagcctataatgggtgaatattaccggtatgatggtaccccattttacaaggcattgtggagggaggcaaaggagtgcttgttcgtcgagccagatgaaggagctcctacacaaggcgttttctggtaccggaacaagtattaa

SEQ ID NO: 5 - protein, 263 aa, Arabidopsis thaliana
MASCSLGVPKIKISAVDLSRVRSGSLLIPYNQRSLLRQRPVKYLSLKTTFGSVKAVQVSTVPTAETSATIEVKDSKEIKSSRLNAQLVPKPSEVEALVTEICDSSSIAEFELKLGGFRLYVARNIADNSSLQPPPTPAVTASNATTESPESNGSASSTSLAISKPASSAADQGLMILQSPKVGFFRRSKTIKGKRLPSSCKEKDQVKEGQILCYIEQLGGQFPIESDVTGEVVKILREDGEPVGYNDALISILPSFPGIKKLQ

SEQ ID NO: 6 - DNA, 2698 bp, Arabidopsis thaliana
accactctggttgtatcgaacgagcgaaacccaaccaacgacgagcgttcacctcaaatattttgatttgatcaaatcatctccacactcgccaaatcgttgtgtcctcgttcatatcgttatcgtatcagctcaaaaatctcaatctctcttccttacattcttctgtttctcgaatccttgtctccctccatggcttcctgtaagtttctctacctgtctcttgttgtcttgcttgttccagttttcttgggatcgtacactaaattgggtttgtgtttcctcattcaaatttgaatgctttcgtagttttctgctctcatagaatcatattcatcgaaaggttgtaactttggggattctgtttattgagtgataggaaaactcagaaagggacttaactttgaacaattaggttgattttggtataaattagagatctaaagttgaagaatttgtcttcagtatctgtttcaatggagatgagattcaagttacttcatttgatattgaattgccaagctaatctaattgatgagtttggcagtgataagttaatttcataatttgtatctcttaatatgaattactcgacaacattacttaatctttcactgttgagtatacgtggagatcggttaacgtgagtttattctaagacattatattttgaattacttaaaactttctggagctatcttggattgagtgtataagatttgcttatgctcaattttaaaaagtgagggatcatattgaagataagtgcttatttagtctttctttttgactctgtcttgttttggctgatttcccatattgagaccttggcgtatgacgtatgttacaggtagcctaggagttcctaaaattaaaatctcggcagtagaccttagtagagtaagatctggaagcttactgataccatacaatcaaagatcattgcttcgacaaaggccagtgaagtacttgagtctgaagacaacatttggatctgtgaaagctgtccaagtgtctactgtcccaactgcagaaacatcaggtacacttatctctatatgttttcttaacttgaatatgctcatttttaccgattttactatcgatatgttttgcacatcgagtgtgttcacatgtgggctgatgtgttcctagaaagtctcttttagttttcctttaatgctttctgatttattcttgttatcaacagctactatagaagtaaaagattctaaagagatcaagtcatctcgattaaacgctcagcttgttcccaagccttctgaggtgggttttgattttccatttaatgttagaatgtcaatttaagaactctggttcttctcccttattgtcaaatggaagagaagaaatgtgttgtcttgaggattaagtggagaattcacttgttgcctgcacaataaaaccatttgagtctgtttttttaattggatgcattcaatatgatttctttttcgatcttttaggtggaagcccttgtaactgaaatatgcgattcttcatcaattgcagagtttgaactgaaagtaaggctctactcaattgaattgttgtcatgttattgctcttttgcagagtcatctcagctaagtttttgaataggattcttatctaataatttcggcctctttcatttgcacattctaatagctggggggtttccgactatatgtagcaaggaacatagctgacaatagtagtctacaacctccgccaactcctgctgtgactgcttcaaatgcaactaccgagagtcctgagtcgaatggatcagcttcctctacttcactggctatctcaaaaccagcatcgtcagccgctgatcagggtttgatgattctccaatctccaaaagtaagagaccacacaactcaaaggcaaaatgtcatatactctgttggaaaatgctatattttatagtttcaatcagaaagttgatcccaatctaaatggtgtgtaatatgtgcaggtagggttcttcaggagatccaaaaccataaagggtaaacgcctgccttcgtcttgtaaagaggtataaccaatcttcttgaacagaagagagtgtttgatttcatgggggaaaccactgactaatctcttatttgctcttgtttaatctgacagaaagaccaagtgaaagaaggtcaaattctgtgctacattgaacaactcggtggccaatttccaatcgaggttagataatattccattttaattcctgatttagtaattactatcacttgcttcaaccaactcagttaaattgcttctctgtttatcgatcaatcttctagtctgatgttaccggcgaggtagtcaagatactccgagaagatggaggcaagtctctcgtcttctttaacctttcttcgtttttcttaaaacctcggtgtaatgatttttcttatcgttttctcattcggaacagagcctgtaggatacaatgatgctctcatctccatccttccatccttccctgggatcaagaagcttcagtaaaaccaaattcgagctggttttgagttatgacactgtgccttgtgtatgcttttagataaagaaacttcattcatatttgtatttgtcttttgcttgtatgaaagttcttctttaagactcttttattctgtatgctttttcttatatataaaaacattatggtatttttttttaatcg

SEQ ID NO: 7 - protein, 387 aa, Ricinus communis
MGGGGRMSTVITSNNSEKKGGSSHLKRAPHTKPPFTLGDLKRAIPPHCSERSFVRSFSYVAYDVCLSFLFYSIATNFFPYISSPLSYVAWLVYWLFQGCILTGLWVIGHECGHHAFSEYQLADDIVGLIVHSALLVPYFSWKYSHRRHHSNIGSLERDEVFVPKSKSKISWYSKYLNNPPGRVLTLAATLLLGWPLYLAFNVSGRPYDRFACHYDPYGPIFSERERLQIYIADLGIFATTFALYQATMAKGLAWVMRIYGVPLLIVNCFLVMITYLQHTHPAIPRYGSSEWDWLRGAMVTVDRDYGVLNKVFHNIADTQVAHHLFATVPHYHAMEATKAIKPIMGEYYRYDGTPFYKALWREAKECLFVEPDEGAPTQGVFWYRNKY

SEQ ID NO: 8 - DNA, 831 bp, Artificial Sequence
  Synthetic
atggcgtcttctgcagctctcggatctcttcatcagactttagggtcacagagtgagcttcatttgctttctggaaactggtctgcctctggtacttcttgcgttccacggtggagattatccaacaggagtagcaattacacgcttgtgttacgtgcaaaggcctctaaaacttcgacaacaaccaaaagcgatgattcatctgatgcgactgtgtcaaacgggaagaaatctgttcgacggacaaccttcccgaaagaagtggaggcactggttcacgagatgtgtgatgagactgaggttgctgtcctgaaacttaaggttggagatttcgagatgaacctaaaacggaagattggagcggccacaaaccccattcctgtggaggatatatctccaaccgtagcacctccgattccttctgagcccatggataaatctgtttcttctgctcccagcccatctaaagcaaaaccgtctgaaaaagtatctccatttatgaatacatcatatgggaaaccagcgaagttggtagctttggaggcatctggatcaaacaattatgttctagtcaaatctccctcagttggcgagtttcacagaagcagaactgtaaaaggaaagaaactatctcctagctgcaaagagggtgatgaaataaaggaaggccaagttattggatacttacatcagttgggaacagaacttccagtgacgtcggatgtagctggggaagtcctcaagcttctttcagatgacggagactccgtaggttatggtgatcctctggttgcggtcttgccatcgttccacgatatcaacatccagtga

SEQ ID NO: 9 - protein, 276 aa, Artificial Sequence
  Synthetic
MASSAALGSLHQTLGSQSELHLLSGNWSASGTSCVPRWRLSNRSSNYTLVLRAKASKTSTTTKSDDSSDATVSNGKKSVRRTTFPKEVEALVHEMCDETEVAVLKLKVGDFEMNLKRKIGAATNPIPVEDISPTVAPPIPSEPMDKSVSSAPSPSKAKPSEKVSPFMNTSYGKPAKLVALEASGSNNYVLVKSPSVGEFHRSRTVKGKKLSPSCKEGDEIKEGQVIGYLHQLGTELPVTSDVAGEVLKLLSDDGDSVGYGDPLVAVLPSFHDINIQ

SEQ ID NO: 10 - DNA, 846 bp, Artificial Sequence
  Synthetic
atggcgtcttctgcagctctcggatctcttcatcagactttagggtcacagagtgagcttcacttgctttctggaaattggtctgcttctggtacttcttgtgtaccacggtggagattatccaacaggagcagcaattacacgcttgtgttacgtgcaaaggcctctaaaacttcgacaacaaccaaaagcgatgattcatctgatgcgactgtgtcaaacgggaagaaatctgttcgacggacaactttcccgaaagaagtggaggcactggttcacgagatgtgtgatgagactgaggttgctgtcctgaaacttaaggcaagttactctggcgttggagatttcgagatgaacctaaaacggaagattgaagcggccacaaaccccattcctgtggaggatatatctccaaccgtagcacctccgattccttctgagcccatgaatcaatcggtttcctctattcctagcccatctaaagcaaaaccttctgaaaaagtatctccatttataaatacatcatatgggaaaccagcaaagttggcagctttggaggcatctggatcaaataattatgttctagtcaaatctccctcagttggcgagtttcacagaagcagaactgtaaaaggaaagaaactatctcctagctgcaaagagggtgatgaaataaaggaagggcaagttattggatacttacatcagttgggaacagaacttccagtgacgtcggatgtagctggggaagtcctcaagcttctttcagatgacggagactccgtaggttatggtgatcctctggttgcggtcttgccatcgttccacgatatcaacatccagtga

SEQ ID NO: 11 - protein, 281 aa, Artificial Sequence
  Synthetic
MASSAALGSLHQTLGSQSELHLLSGNWSASGTSCVPRWRLSNRSSNYTLVLRAKASKTSTTTKSDDSSDATVSNGKKSVRRTTFPKEVEALVHEMCDETEVAVLKLKASYSGVGDFEMNLKRKIEAATNPIPVEDISPTVAPPIPSEPMNQSVSSIPSPSKAKPSEKVSPFINTSYGKPAKLAALEASGSNNYVLVKSPSVGEFHRSRTVKGKKLSPSCKEGDEIKEGQVIGYLHQLGTELPVTSDVAGEVLKLLSDDGDSVGYGDPLVAVLPSFHDINIQ

SEQ ID NO: 12 - protein, 891 aa, Artificial Sequence
  Synthetic
ATGGCGTCTTCTGCAGCTCTCGGATCTCTTCATCATCCGATCTTTTTGTGGCAATTGGTTGTTGTGGTGACTGAATTAGAGACTTTAGGGTCACAGAGTGAGCTTCACTTGCTTTCTGGAAATTGGTCTGCTTCTGGTACTTCTTGTGTACCACGGTGGAGATTATCCAACAGGAGCAGCAATTACACGCTTGTGTTACGTGCAAAGGCCTCTAAAACTTCGACAACAACCAAAAGCGATGATTCATCTGATGCAACTGTGTCAAACGGGAAGAAATCTGTTCGAAGGACAACTTTCCCGAAAGAAGTGGAGACACTGGTTCACGAGATGTGTGATGAGACTGAGGTTGCTGTCCTGAAACTCAAGGCAAGATACTCTGGCGTTGGAGATTTCGAGATGAACCTAAAACGGAAGATTGGAGCTACCACAAACCCCATTCCTGTGGAGGATATATCTCCAACCGTAGCACCTCCAATTCCTTCTGAGCCCATGAATCAATCGGTTTCCTCTGCTCCCAGCCCATCTACAGCAAAACCGTCTGAAAAAGTATCTCCATTTATGAATACATCATATGGGAAACCAGCAAAGTTGGCAGCTTTGGAGGCATCTGGATCAAACAATTATGTTCTAGTCAAATCTCCCTCAGTTGGCGAGTTTCACAGAAGCAGAACTGTAAAAGGAAAGAAACTATCTCCTAGCTGCAAAGAGGGTGATGAAATAAAGGAAGGCCAAGTGATTGGATACTTACATCAGTTGGGAACAGAACTTCCAGTGACGTCGGATGTAGCTGGGGAAGTCCTCAAGCTTCTTTCAGATGACGGAGACTCCATAGGTTATGGTGATCCTCTGGTTGCGGTCTTGCCATCGTTCCACGATATCAACATCCAGTGA

SEQ ID NO: 13 - protein, 296 aa, Artificial Sequence
  Synthetic
MASSAALGSLHHPIFLWQLVVVVTELETLGSQSELHLLSGNWSASGTSCVPRWRLSNRSSNYTLVLRAKASKTSTTTKSDDSSDATVSNGKKSVRRTTFPKEVETLVHEMCDETEVAVLKLKARYSGVGDFEMNLKRKIGATTNPIPVEDISPTVAPPIPSEPMNQSVSSAPSPSTAKPSEKVSPFMNTSYGKPAKLAALEASGSNNYVLVKSPSVGEFHRSRTVKGKKLSPSCKEGDEIKEGQVIGYLHQLGTELPVTSDVAGEVLKLLSDDGDSIGYGDPLVAVLPSFHDINIQ

SEQ ID NO: 14 - DNA, 792 bp, Artificial Sequence
  Synthetic
atggcttcctgtagcctaggagttcctaaaattaaaatctcagcagtagaccttagtagagtaagttctggaagcttactgataccattcagccaaagatcattgcttggacaaaggccggtgaagtacttgagtctcaggacaacttttggatctgtgaaagctgtccaagtatctactgtcccaaccgcagaaacatcagctactatagaagtagaagattctgaagaaaccaagtcatctccattgaacgctcagctagttcccaagccatctgaggtggaagctcttgtcactgaaatatgcgattcctcatcaattgcagagtttgaattgaaactggggggtttccgcctatatgtagcaagggatctaactgacaaaagtagtccgcagcctcatccagttcctgctgtggctgctgccagtgaaactaccaagagtcctgattcgaatggatcaactccttctacttcattggctatcacaagaccagcatcctcagctgctgatcacggtttgatgattctccaatctccaaaagtagggttcttcaggagatccaaaactataaagggtaaacgcatgccttcgtcatgtaaagagaaagaccaagtgaaagaaggtcaaattctgtgctacattgaacaactcggtggccaattcccaatagagtctgatgtcagcggcgaggttgtcaaaatactccgagaagatggagagcctgtaggatacaatgatgctctcatctcgatccttccctctttccctgggatcaagaagcttcagtaa

SEQ ID NO: 15 - protein, 263 aa, Artificial Sequence
  Synthetic
MASCSLGVPKIKISAVDLSRVSSGSLLIPFSQRSLLGQRPVKYLSLRTTFGSVKAVQVSTVPTAETSATIEVEDSEETKSSPLNAQLVPKPSEVEALVTEICDSSSIAEFELKLGGFRLYVARDLTDKSSPQPHPVPAVAAASETTKSPDSNGSTPSTSLAITRPASSAADHGLMILQSPKVGFFRRSKTIKGKRMPSSCKEKDQVKEGQILCYIEQLGGQFPIESDVSGEVVKILREDGEPVGYNDALISILPSFPGIKKLQ

SEQ ID NO: 16 - DNA, 792 bp, Artificial Sequence
  Synthetic
atggcatcctgtagcctaggagttcctaaaattaaaatctcagcagtagaccttagtagagtaagttctggaagcttactgataccattcagtcaaagatcattgcttggacaaaggccggtgaagtacttgagtctgaggacaacttttggatctgtgaaagctgtacaagtatctactgtcccagctgcagaaacatcagctactgtaggagtagaagattctgaagaaaccaagtcatccccattgaacgctcagctagttcccaagcgatctgaggtggaagctcttgtcactgaaatatgcgactcctcatcaattgcagagtttgaactgaaactggggggtttccgcctatatgtagcaagggatctagctgacaaaagtagtccgcagcctcatccaattcctgctgtggctgctgcaagtgaaactaccaagagtcctgattcgaatggatcaacaccttctacttcattggctatcacaagaccagcatcttcagctgctgatcagggtttgatgattctccaatctccaaaagtagggttctttaggagatccaaaaccataaagggtaaacgcatgccttcgtcatgtaaagagaaagaccaagtgaaagaaggtcaaattctgtgctacattgaacaactcggtggccaattcccaatagagtctgatgtcagcggtgaggttgtcaaaatactccgcgaagatggagaacctgtaggatacaatgatgctctcatctcgatccttccctctttccctgggatcaagaagcttcagtaa

SEQ ID NO: 17 - protein, 263 aa, Artificial Sequence
  Synthetic
MASCSLGVPKIKISAVDLSRVSSGSLLIPFSQRSLLGQRPVKYLSLRTTFGSVKAVQVSTVPAAETSATVGVEDSEETKSSPLNAQLVPKRSEVEALVTEICDSSSIAEFELKLGGFRLYVARDLADKSSPQPHPIPAVAAASETTKSPDSNGSTPSTSLAITRPASSAADQGLMILQSPKVGFFRRSKTIKGKRMPSSCKEKDQVKEGQILCYIEQLGGQFPIESDVSGEVVKILREDGEPVGYNDALISILPSFPGIKKLQ

SEQ ID NO: 18 - DNA, 1005 bp, Artificial Sequence
  Synthetic
tgcttcgctgtacaaagaccactctggttgtatcgaacagagcgaacccaaccaacgacgagcgttaacctcaatttgatcatctccacactcgtcaaatctttggtgtcctcctcctcctcgttcgtatcgttatcgtatcagctcacaaatctcattctcttccttacattcttcttcttctgcttctcgaatcctcctttgtctccctccatggcttcctgtagcctaggagttcctaaaattaaaatctcagcagtagaccttagtagagtaagttctggaagcttgctggtaccattcagtcaaagatcattgcttggacaaaggacggtgaagtacttgagtctgaggaaaacttttggatctgtgaaagctgtacaactatctactgtcccagctgcagaaacatcagctactgtaggagtagaagattctgaagaaaccaagtcatctccattgaacgctcagctagttcccaatccatctgaggtggaagctcttgtcactgaaatatgcgactcctcatcaattgcagagtttgaactgaaactggggggtttccgcctatatgtagcaagggatctagctgacaaaagtagtccgcagcctcatccaattcctgctgtggctgctgcaagtgaaactaccaagagtcctgattcgaatggatcaacaccttctacttcattggctatcacaagaccagcatcttcagctgctgatcagggtttgatgattctccaatctccaaaagtagggttctttaggagatccaaaaccataaagggtaaacgcatgccttcgtcatgtaaagagaaagaccaagtgaaagaaggtcaaattctgtgctacattgaacaactcggtggccaattcccaatagagtctgatgtcagcggcgaggttgtcaaaatactccgagaagatggagagcctgtagggtacaatgatgctctcatctcgatccttccctctttccctgggatcaagaagcttcagtaa

SEQ ID NO: 19 - protein, 263 aa, Artificial Sequence
  Synthetic
MASCSLGVPKIKISAVDLSRVSSGSLLVPFSQRSLLGQRTVKYLSLRKTFGSVKAVQLSTVPAAETSATVGVEDSEETKSSPLNAQLVPNPSEVEALVTEICDSSSIAEFELKLGGFRLYVARDLADKSSPQPHPIPAVAAASETTKSPDSNGSTPSTSLAITRPASSAADQGLMILQSPKVGFFRRSKTIKGKRMPSSCKEKDQVKEGQILCYIEQLGGQFPIESDVSGEVVKILREDGEPVGYNDALISILPSFPGIKKLQ

SEQ ID NO: 20 - DNA, 1521 bp, Artificial Sequence; identical to SEQ ID NO: 3
  Synthetic

SEQ ID NO: 21 - protein, 506 aa, Artificial Sequence
  Synthetic
MTSVNVKLLYRYVLTNFFNLCLFPLTAFLAGKASRLTINDLHNFLSYLQHNLITVTLLFAFTVFGLVLYIVTRPNPVYLVDYSCYLPPPHLKVSVSKVMDIFYQIRKADTSSRNVACDDPSSLDFLRKIQERSGLGDETYSPEGLIHVPPRKTFAASREETEKVIIGALENLFENTKVNPREIGILVVNSSMFNPTPSLSAMVVNTFKLRSNIKSFNLGGMGCSAGVIAIDLAKDLLHVHKNTYALVVSTENITQGIYAGENRSMMVSNCLFRVGGAAILLSNKSGDRRRSKYKLVHTVRTHTGADDKSFRCVQQEDDESGKIGVCLSKDITNVAGTTLTKNIATLGPLILPLSEKFLFFATFVAKKLLKDKIKHYYVPDFKLAVDHFCIHAGGRAVIDELEKNLGLSPIDVEASRSTLHRFGNTSSSSIWYELAYIEAKGRMKKGNKAWQIALGSGFKCNSAVWVALRNVKASANSPWQHCIDRYPVKIDSDLSKSKTHVQNGRS

SEQ ID NO: 22 - protein, 1152 aa, Artificial Sequence
  Synthetic
ATGGGTGCAGGTGGAAGAATGCCGGTTCCTACTTCTTCCAAGAAATCGGAAACCGACACCACAAAGCGTGTGCCGTGCGAGAAACCGCCTTTCTCGGTGGGAGATCTGAAGAAAGCAATCCCGCCGCATTGTTTCAAACGCTCAATCCCTCGCTCTTTCTCCTACCTTATCAGTGACATCATTATAGCCTCATGCTTCTACTACGTCGCCACCAATTACTTCTCTCTCCTCCCTCAGCCTCTCTCTTACTTGGCTTGGCCACTCTATTGGGCCTGTCAAGGCTGTGTCCTAACTGGTATCTGGGTCATAGCCCACGAATGCGGTCACCACGCATTCAGCGACTACCAATGGCTGGATGACACAGTTGGTCTTATCTTCCATTCCTTCCTCCTCGTCCCTTACTTCTCCTGGAAGTATAGTCATCGCCGTCACCATTCCAACACTGGATCCCTCGAAAGAGATGAAGTATTTGTCCCAAAGCAGAAATCAGCAATCAAGTGGTACGGGAAATACCTCAACAACCCTCTTGGACGCATCATGATGTTAACCGTCCAGTTTGTCCTCGGGTGGCCCTTGTACTTAGCCTTTAACGTCTCTGGCAGACCGTATGACGGGTTCGCTTGCCATTTCTTCCCCAACGCTCCCATCTACAATGACCGAGAACGCCTCCAGATATACCTCTCTGATGCGGGTATTCTAGCCGTCTGTTTTGGTCTTTACCGTTACGCTGCTGCACAAGGGATGGCCTCGATGATCTGCCTCTACGGAGTACCGCTTCTGATAGTGAATGCGTTCCTCGTCTTGATCACTTACTTGCAGCACACTCATCCCTCGTTGCCTCACTACGATTCATCAGAGTGGGACTGGCTCAGGGGAGCTTTGGCTACCGTAGACAGAGACTACGGAATCTTGAACAAGGTGTTCCACAACATTACAGACACACACGTGGCTCATCACCTGTTCTCGACAATGCCGCATTATAACGCAATGGAAGCTACAAAGGCGATAAAGCCAATTCTGGGAGACTATTACCAGTTCGATGGAACACCGTGGTATGTAGCGATGTATAGGGAGGCAAAGGAGTGTATCTATGTAGAACCGGACAGGGAAGGTGACAAGAAAGGTGTGTACTGGTACAACAATAAGTTATGA

SEQ ID NO: 23 - protein, 383 aa, Artificial Sequence
  Synthetic
MGAGGRMPVPTSSKKSETDTTKRVPCEKPPFSVGDLKKAIPPHCFKRSIPRSFSYLISDIIIASCFYYVATNYFSLLPQPLSYLAWPLYWACQGCVLTGIWVIAHECGHHAFSDYQWLDDTVGLIFHSFLLVPYFSWKYSHRRHHSNTGSLERDEVFVPKQKSAIKWYGKYLNNPLGRIMMLTVQFVLGWPLYLAFNVSGRPYDGFACHFFPNAPIYNDRERLQIYLSDAGILAVCFGLYRYAAAQGMASMICLYGVPLLIVNAFLVLITYLQHTHPSLPHYDSSEWDWLRGALATVDRDYGILNKVFHNITDTHVAHHLFSTMPHYNAMEATKAIKPILGDYYQFDGTPWYVAMYREAKECIYVEPDREGDKKGVYWYNNKL

SEQ ID NO: 24 - DNA, 1161 bp, Artificial Sequence
  Synthetic
atggttgttgctatggaccaacgcaccaatgtgaacggagatcccggcgccggagaccggaagaaagaagaaaggtttgatccgagtgcacaaccaccgttcaagatcggagatataagggcggcgattcctaagcactgttgggttaagagtcctttgagatcaatgagttacgtcgtcagagacattatcgccgtcgcggctttggccatcgctgccgtgtatgttgatagctggttcctttggcctctttattgggccgcccaaggaacacttttctgggccatctttgttctcggccacgactgtggacatgggagtttctcagacattcctctactgaatagtgtggttggtcacattcttcattctttcatcctcgttccttaccatggttggagaataagccaccggacacaccaccagaaccatggccatgttgaaaacgacgagtcatgggttccgttaccagaaagggtgtacaagaaattgccccacagtactcggatgctcagatacactgtccctctccccatgctcgcatatcctctctatttgtgctacagaagtcctggaaaagaaggatcacattttaacccatacagtagtttatttgctccaagcgagagaaagcttattgcaacttcaactacttgttggtccataatgttcgtcagtcttatcgctctatctttcgtcttcggtccactcgcggttcttaaagtctacggtgtaccgtacattatctttgtgatgtggttggatgctgtcacgtatttgcatcatcatggtcacgatgagaagttgccttggtatagaggcaaggaatggagttatctacgtggaggattaacaacaattgatagagattacggaatctttaacaacattcatcacgacattggaactcacgtgatccatcatctcttcccacaaatccctcactatcacttggtcgacgccacgaaagcagctaaacatgtgttgggaagatactacagagaaccaaagacgtcaggagcaataccgatccacttggtggagagtttggtcgcaagtattaagaaagatcattacgtcagcgacactggtgatattgtcttctacgagacagatccagatctctacgtttacgcttctgacaaatctaaaatcaattaa

SEQ ID NO: 25 - protein, 386 aa, Artificial Sequence
  Synthetic
MVVAMDQRTNVNGDPGAGDRKKEERFDPSAQPPFKIGDIRAAIPKHCWVKSPLRSMSYVVRDIIAVAALAIAAVYVDSWFLWPLYWAAQGTLFWAIFVLGHDCGHGSFSDIPLLNSVVGHILHSFILVPYHGWRISHRTHHQNHGHVENDESWVPLPERVYKKLPHSTRMLRYTVPLPMLAYPLYLCYRSPGKEGSHFNPYSSLFAPSERKLIATSTTCWSIMFVSLIALSFVFGPLAVLKVYGVPYIIFVMWLDAVTYLHHHGHDEKLPWYRGKEWSYLRGGLTTIDRDYGIFNNIHHDIGTHVIHHLFPQIPHYHLVDATKAAKHVLGRYYREPKTSGAIPIHLVESLVASIKKDHYVSDTGDIVFYETDPDLYVYASDKSKIN

SEQ ID NO: 26 - DNA, 1521 bp, Artificial Sequence
  Synthetic
atgacgtccgttaacgttaagctcctttaccgttacgtcttaaccaactttttcaacctctgtttgttcccgttaacggcgttcctcgccggaaaagcctctcggcttaccataaacgatctccacaacttcctttcctatctccaacacaaccttataacagtaactttactctttgctttcactgttttcggtttggttctctacatcgtaacccgacccaatccggtttatctcgttgactactcgtgttaccttccaccaccgcatctcaaagttagtgtctctaaagtcatggatattttctaccaaataagaaaagctgatacttcttcacggaacgtggcatgtgatgatccgtcctcgctcgatttcctgaggaagattcaagagcgttcaggtctaggtgatgagacgtacagtcctgagggactcattcacgtaccaccgcggaagacttttgcagcgtcacgtgaagagacagagaaggttatcatcggtgcgctcgaaaatctattcgagaacaccaaagttaaccctagagagattggtatacttgtggtgaactcaagcatgtttaatccaactccttcgctatccgctatggtcgttaatactttcaagctccgaagcaacatcaaaagctttaatctaggaggaatgggttgtagtgctggtgttattgccattgatttggctaaagacttgttgcatgttcataaaaacacttatgctcttgtggtgagcactgagaacatcacacaaggcatttatgctggagaaaatagatcaatgatggttagcaattgcttgtttcgtgttggtggggccgcgattttgctctctaacaagtcgggagaccggagacggtccaagtacaagctagttcacacggtccgaacgcatactggagctgatgacaagtcttttcgatgtgtgcaacaagaagacgatgagagcggcaaaatcggagtttgtctgtcaaaggacataaccaatgttgcggggacaacacttacgaaaaatatagcaacattgggtccgttgattcttcctttaagcgaaaagtttctttttttcgctaccttcgtcgccaagaaacttctaaaggataaaatcaagcattactatgttccggatttcaagcttgctgttgaccatttctgtattcatgccggaggcagagccgtgatcgatgagctagagaagaacttaggactatcgccgatcgatgtggaggcatctagatcaacgttacatagatttgggaatacttcatctagctcaatttggtatgaattagcatacatagaggcaaagggaagaatgaagaaagggaataaagcttggcagattgctttaggatcagggtttaagtgtaatagtgcggtttgagtggctctacgcaatgtcaaggcatcggcaaatagtccttggcaacattgcatcgatagatatccggttaaaattgattctgatttgtcaaagtcaaagactcatgtccaaaacggtcggtcctaa

SEQ ID NO: 27 - protein, 464 aa, Artificial Sequence
  Synthetic
MTSVNVKLLYRYVLTNFFNLCLFPLTAFLAGKASRLTINDLHNFLSYLQHNLITVTLLFAFTVFGLVLYIVTRPNPVYLVDYSCYLPPPHLKVSVSKVMDIFYQIRKADTSSRNVACDDPSSLDFLRKIQERSGLGDETYSPEGLIHVPPRKTFAASREETEKVIIGALENLFENTKVNPREIGILVVNSSMFNPTPSLSAMVVNTFKLRSNIKSFNLGGMGCSAGVIAIDLAKDLLHVHKNTYALVVSTENITQGIYAGENRSMMVSNCLFRVGGAAILLSNKSGDRRRSKYKLVHTVRTHTGADDKSFRCVQQEDDESGKIGVCLSKDITNVAGTTLTKNIATLGPLILPLSEKFLFFATFVAKKLLKDKIKHYYVPDFKLAVDHFCIHAGGRAVIDELEKNLGLSPIDVEASRSTLHRFGNTSSSSIWYELAYIEAKGRMKKGNKAWQIALGSGFKCNSAV

SEQ ID NO: 28 - DNA, 1149 bp, Escherichia coli
atgagttcatcgtgtatagaagaagtcagtgtaccggatgacaactggtaccgtatcgccaacgaattacttagccgtgccggtatagccattaacggttctgccccggcggatattcgtgtgaaaaaccccgatttttttaaacgcgttctgcaagaaggctctttggggttaggcgaaagttatatggatggctggtgggaatgtgaccgactggatatgttttttagcaaagtcttacgcgcaggtctcgagaaccaactcccccatcatttcaaagacacgctgcgtattgccggcgctcgtctcttcaatctgcagagtaaaaaacgtgcctggatagtcggcaaagagcattacgatttgggtaatgacttgttcagccgcatgcttgatcccttcatgcaatattcctgcgcttactggaaagatgccgataatctggaatctgcccagcaggcgaagctcaaaatgatttgtgaaaaattgcagttaaaaccagggatgcgcgtactggatattggctgcggctggggcggactggcacactacatggcatctaattatgacgtaagcgtggtgggcgtcaccatttctgccgaacagcaaaaaatggctcaggaacgctgtgaaggcctggatgtcaccattttgctgcaagattatcgtgacctgaacgaccagtttgatcgtattgtttctgtggggatgttcgagcacgtcggaccgaaaaattacgatacctattttgcggtggtggatcgtaatttgaaaccggaaggcatattcctgctccatactatcggttcgaaaaaaaccgatctgaatgttgatccctggattaataaatatatttttccgaacggttgcctgccctctgtacgccagattgctcagtccagcgaaccccactttgtgatggaagactggcataacttcggtgctgattacgatactacgttgatggcgtggtatgaacgattcctcgccgcatggccagaaattgcggataactatagtgaacgctttaaacgaatgtttacctattatctgaatgcctgtgcaggtgctttccgcgcccgtgatattcagctctggcaggtcgtgttctcacgcggtgttgaaaacggccttcgagtggctcgctaa

SEQ ID NO: 29 - protein, 382 aa, Escherichia coli
MSSSCIEEVSVPDDNWYRIANELLSRAGIAINGSAPADIRVKNPDFFKRVLQEGSLGLGESYMDGWWECDRLDMFFSKVLRAGLENQLPHHFKDTLRIAGARLFNLQSKKRAWIVGKEHYDLGNDLFSRMLDPFMQYSCAYWKDADNLESAQQAKLKMICEKLQLKPGMRVLDIGCGWGGLAHYMASNYDVSVVGVTISAEQQKMAQERCEGLDVTILLQDYRDLNDQFDRIVSVGMFEHVGPKNYDTYFAVVDRNLKPEGIFLLHTIGSKKTDLNVDPWINKYIFPNGCLPSVRQIAQSSEPHFVMEDWHNFGADYDTTLMAWYERFLAAWPEIADNYSERFKRMFTYYLNACAGAFRARDIQLWQVVFSRGVENGLRVAR

SEQ ID NO: 30 - DNA, 1125 bp, Crepis palaestina
atgggtgccggcggtcgtggtcggacatcggaaaaatcggtcatggaacgtgtctcagttgatccagtaaccttctcactgagtgaattgaagcaagcaatccctccccattgcttccagagatctgtaatccgctcatcttactatgttgttcaagatctcattattgcctacatcttctacttccttgccaacacatatatccctactcttcctactagtctagcctacttagcttggcccgtttactggttctgtcaagctagcgtcctcactggcttatggatcctcggccacgaatgtggtcaccatgcctttagcaactacacatggtttgacgacactgtgggcttcatcctccactcatttctcctcaccccgtatttctcttggaaattcagtcaccggaatcaccattccaacacaagttcgattgataacgatgaagtttacattccgaaaagcaagtccaaactcgcgcgtatctataaacttcttaacaacccacctggtcggctgttggttttgattatcatgttcaccctaggatttcctttatacctcttgacaaatatttccggcaagaaatacgacaggtttgccaaccacttcgaccccatgagtccaattttcaaagaacgtgagcggtttcaggtcttcctttcggatcttggtcttcttgccgtgttttatggaattaaagttgctgtagcaaataaaggagctgcttgggtagcgtgcatgtatggagttccggtattaggcgtatttacctttttcgatgtgatcaccttcttgcaccacacccatcagtcgtcgcctcattatgattcaactgaatggaactggatcagaggggccttgtcagcaatcgatagggactttggattcctgaatagtgttttccatgatgttacacacactcatgtcatgcatcatttgttttcatacattccacactatcatgcaaaggaggcaagggatgcaatcaagccaatcttgggcgacttttatatgatcgacaggactccaattttaaaagcaatgtggagagagggcagggagtgcatgtacatcgagcctgatagcaagctcaaaggtgtttattggtatcataaattgtga

SEQ ID NO: 31 - protein, 374 aa, Crepis palaestina
MGAGGRGRTSEKSVMERVSVDPVTFSLSELKQAIPPHCFQRSVIRSSYYVVQDLIIAYIFYFLANTYIPTLPTSLAYLAWPVYWFCQASVLTGLWILGHECGHHAFSNYTWFDDTVGFILHSFLLTPYFSWKFSHRNHHSNTSSIDNDEVYIPKSKSKLARIYKLLNNPPGRLLVLIIMFTLGFPLYLLTNISGKKYDRFANHFDPMSPIFKERERFQVFLSDLGLLAVFYGIKVAVANKGAAWVACMYGVPVLGVFTFFDVITFLHHTHQSSPHYDSTEWNWIRGALSAIDRDFGFLNSVFHDVTHTHVMHHLFSYIPHYHAKEARDAIKPILGDFYMIDRTPILKAMWREGRECMYIEPDSKLKGVYWYHKL

SEQ ID NO: 32 - DNA, 1131 bp, Crepis alpina
aagatgggtggcggtggccgtggtcggacttcgcaaaaacccctcatggaacgtgtctcagttgatccacccttcaccgtgagtgatctcaagcaagcaatccctccccattgcttcaagcgatctgtaatccgttcctcttactacatagtccacgatgctattatcgcctacatcttctacttccttgccgacaaatacattccgattctccctgcccctctagcctacctcgcttggcccctttactggttctgtcaagctagcatcctcaccggcttatgggtcatcggtcacgaatgcggtcaccatgccttcagcgactaccagtgggttgacgacactgtgggcttcatcctccactcgtttctcatgaccccgtatttctcctggaaatacagccaccggaaccaccatgccaacacaaattcgcttgacaacgatgaagtttacatccccaaaagcaaggccaaagtcgcgctttactataaagttctcaaccacccacctggccgactgttgattatgttcatcaccttcaccctaggcttccctctatacctctttaccaatatttccggcaagaagtatgaaaggtttgccaaccatttcgaccccatgagtccgattttcaaagagcgtgagcggtttcaggtcttgctatcggatcttggccttcttgctgtgctttacggagttaaacttgcggtagcagcgaaaggcgccgcttgggtgacgtgcatttacggaattccagttttaggcgtgtttatctttttcgatatcatcacctacttgcaccacacccatctgtcgttgcctcattatgattcatctgaatggaactggctcagaggggctttgtcaacaatcgatagggactttgggttcctgaatagtgtgctccatgatgttacacacactcacgttatgcatcatctgttttcatacattccacactatcatgcgaaggaggcaagggatgcaatcaacacagtcttgggcgacttttataagatcgataggactccaattctgaaagcaatgtggagagaggccaaggaatgcatcttcatcgagcctgaaaaaggtagggagtccaagggtgtatattggtacaataaattctga

SEQ ID NO: 33 - protein, 375 aa, Crepis alpina
MGGGGRGRTSQKPLMERVSVDPPFTVSDLKQAIPPHCFKRSVIRSSYYIVHDAIIAYIFYFLADKYIPILPAPLAYLAWPLYWFCQASILTGLWVIGHECGHHAFSDYQWVDDTVGFILHSFLMTPYFSWKYSHRNHHANTNSLDNDEVYIPKSKAKVALYYKVLNHPPGRLLIMFITFTLGFPLYLFTNISGKKYERFANHFDPMSPIFKERERFQVLLSDLGLLAVLYGVKLAVAAKGAAWVTCIYGIPVLGVFIFFDIITYLHHTHLSLPHYDSSEWNWLRGALSTIDRDFGFLNSVLHDVTHTHVMHHLFSYIPHYHAKEARDAINTVLGDFYKIDRTPILKAMWREAKECIFIEPEKGRESKGVYWYNKF

SEQ ID NO: 34 - DNA, 1297 bp, Momordica charantia
aataaattagcttctttttttaagtgagtgaagggagatctggaggcaatggggggcagaggagctattggagtactgaggaacggtggcggcccaaaaaagaaaatggggccggggcaggggctggggccgggggagcgcattacacatgccaggcctcccttcagcatcagccagatcaagaaggccattcccccccactgctttcagcgatccctccgccgctctttttcctaccttctttccgacattgccctcgtctctgccttttattacgttgccgacacctacttccaccgcctgccccaccccctactccactacctggcctggcccgtttactggttctgtcagggcgccgtactcaccggcatgtggggcatcgctcacgactgcggccaccacgccttcagcgactaccaattggtagacgacgtggttgggttcctcatccactctttggtttttgtcccttacttctccttcaagatcagccaccgccgccaccactccaacacctcatccgtggaccgggacgaggtgttcgtccccaagccgaaggccaaaatgccctggtacttcaagtacttgacaaacccgcccgccagggtcttcattatttttatcacgctcactctcgggtggccaatgtacctgaccttcaacatctccggccggtactacggccggttcaccagccacttcgacccgaacagccccatattcagcccaaaggagcgcgttctcgttcatatctccaacgctgggcttgtggcgaccgggtatttgctgtacaggatcgcaatggcgaagggggtggggtggttgatccgcttgtacggagtgccgctgatcgttttaaacgcgtgcgtagttctgatcacagcgctgcagcacacccacccttcgttcccgtattacgactcgacggaatgggattggctgagagggaatctggtgacggtggacagagattacgggcctataatgaatagagtgtttcatcacataacggacacgcacgtggttcaccatttgtttccttcgatgccgcactacaacgggaaagaggcgacggttgcagcaaagcgaatactgggagagtactaccagtttgatgggaccccaatttggaaggcggcctggagggaattcagagagtgcgtttatgtagagccagacgaagacgatggggccacttccggctccagtagtaagggtgttttctggtaccacaacaagctctgaattcaataatatcctctttcacctctctttttcataaaaaaaaaaaaaa

SEQ ID NO: 35 - protein, 399 aa, Momordica charantia
MGGRGAIGVLRNGGGPKKKMGPGQGLGPGERITHARPPFSISQIKKAIPPHCFQRSLRRSFSYLLSDIALVSAFYYVADTYFHRLPHPLLHYLAWPVYWFCQGAVLTGMWGIAHDCGHHAFSDYQLVDDVVGFLIHSLVFVPYFSFKISHRRHHSNTSSVDRDEVFVPKPKAKMPWYFKYLTNPPARVFIIFITLTLGWPMYLTFNISGRYYGRFTSHFDPNSPIFSPKERVLVHISNAGLVATGYLLYRIAMAKGVGWLIRLYGVPLIVLNACVVLITALQHTHPSFPYYDSTEWDWLRGNLVTVDRDYGPIMNRVFHHITDTHVVHHLFPSMPHYNGKEATVAAKRILGEYYQFDGTPIWKAAWREFRECVYVEPDEDDGATSGSSSKGVFWYHNKL

SEQ ID NO: 36 - DNA, 26 bp, Artificial Sequence
  Synthetic
catgagtttgagtatacacatgtcta

SEQ ID NO: 37 - DNA, 31 bp, Artificial Sequence
  Synthetic
aaagaaatcatgtaaacctaaatagaaacgc

SEQ ID NO: 38 - DNA, 24 bp, Artificial Sequence
  Synthetic
gtgatcgatgagctagagaagaac

SEQ ID NO: 39 - DNA, 41 bp, Artificial Sequence
  Synthetic
caaggactatttgccgatgccttgacattgcgtagagcgac

SEQ ID NO: 40 - DNA, 24 bp, Artificial Sequence
  Synthetic
aatatagccatcgccgccaccatt

SEQ ID NO: 41 - DNA, 24 bp, Artificial Sequence
  Synthetic
tggcaagcaaagcgatcgtaaggt

SEQ ID NO: 42 - DNA, 20 bp, Artificial Sequence
  Synthetic
accatcactttggaggtgga

SEQ ID NO: 43 - DNA, 20 bp, Artificial Sequence
  Synthetic
gtcaatggtgtcggagcttt

SEQ ID NO: 44 - DNA, 20 bp, Artificial Sequence
  Synthetic
cggtggagattatccaacag

SEQ ID NO: 45 - DNA, 20 bp, Artificial Sequence
  Synthetic
ttatggtgatcctctggttg

SEQ ID NO: 46 - DNA, 20 bp, Artificial Sequence
  Synthetic
aaaattaaaatctcagcagt